(19) United States
(12) Patent Application Publication
(10) Pub. No.: US 2024/0321453 A1
(43) Pub. Date: Sep. 26, 2024

(54) INFORMATION PROCESSING APPARATUS, INFORMATION PROCESSING SYSTEM, AND INFORMATION PROCESSING METHOD

(71) Applicants: Kazuki FUNAHASHI, Kanagawa (JP); Toshiaki TOKITA, Kanagawa (JP); Masako YOSHII, Kanagawa (JP); Naoki SAKAI, Kanagawa (JP)

(72) Inventors: Kazuki FUNAHASHI, Kanagawa (JP); Toshiaki TOKITA, Kanagawa (JP); Masako YOSHII, Kanagawa (JP); Naoki SAKAI, Kanagawa (JP)

(21) Appl. No.: 18/601,026

(22) Filed: Mar. 11, 2024

(30) Foreign Application Priority Data
Mar. 22, 2023 (JP) 2023-046016

(51) Int. Cl. G16H 50/20; G16H 20/70; G16H 40/20

(52) U.S. Cl. CPC G16H 50/20; G16H 20/70; G16H 40/20

(57) ABSTRACT

An information processing apparatus includes circuitry. The circuitry acquires first information from an answer to an inquiry to a user. The answer has been input to a terminal apparatus used by the user. The first information indicates motivation of the user. The circuitry further acquires second information from the inquiry with the terminal apparatus. The second information indicates a condition related to health of the user. The circuitry calculates a plurality of indicators associated with an organization based on the first information and the second information, and outputs the plurality of indicators.

### CROSS-REFERENCE TO RELATED APPLICATION

This patent application is based on and claims priority pursuant to 35 U.S.C. § 119(a) to Japanese Patent Application No. 2023-046016, filed on Mar. 22, 2023, in the Japan Patent Office, the entire disclosure of which is hereby incorporated by reference herein.

### BACKGROUND

### Technical Field

Embodiments of this disclosure relate to an information processing apparatus, an information processing system, and an information processing method.

### Related Art

To predict an improvement or decline in the performance of personnel, there is a work support system that derives a predicted performance value based on attendance and absence information and medical checkup information of the personnel.

This technique visualizes indicators based on the attendance and absence information and the medical checkup information but not in consideration of the motivation of users.

### SUMMARY

In one embodiment of this invention, there is provided an information processing apparatus that includes, for example, circuitry that acquires first information from an answer to an inquiry to a user. The answer has been input to a terminal apparatus used by the user. The first information indicates motivation of the user. The circuitry further acquires second information from the inquiry with the terminal apparatus. The second information indicates a condition related to health of the user. The circuitry calculates a plurality of indicators associated with an organization based on the first information and the second information, and outputs the plurality of indicators.

In one embodiment of this invention, there is provided an information processing system that includes, for example, a terminal apparatus used by a user and an information processing apparatus communicable with the terminal apparatus. The terminal apparatus includes first circuitry. The information processing apparatus includes second circuitry.

The first circuitry transmits first information to the information processing apparatus. The first information indicates motivation of the user and is acquired from an answer to an inquiry to the user. The answer has been input to the terminal apparatus. The second circuitry acquires the first information from the terminal apparatus, and acquires second information from the inquiry with the terminal apparatus. The second information indicates a condition related to health of the user. The second circuitry calculates a plurality of indicators associated with an organization based on the first information and the second information, and outputs the plurality of indicators.

In one embodiment of this invention, there is provided an information processing method performed by a computer. The information processing method includes, for example, acquiring, from an answer to an inquiry to a user, having been input to a terminal apparatus, first information indicating motivation of the user, acquiring, from the inquiry with the terminal apparatus, second information indicating a condition related to health of the user, calculating a plurality of indicators associated with an organization based on the first information and the second information, and outputting the plurality of indicators.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram illustrating an exemplary system configuration of an information processing system according to a first embodiment of the present invention;

FIG. 2 is a diagram illustrating an exemplary hardware configuration of a computer included in the information processing system of the first embodiment;

FIG. 3 is a diagram illustrating an exemplary hardware configuration of an information terminal included in the information processing system of the first embodiment;

FIG. 4 is a diagram illustrating an exemplary functional configuration of the information processing system of the first embodiment;

FIG. 5 is a flowchart illustrating an example of a survey process of the first embodiment;

FIG. 6 is a diagram illustrating a conceptual example of a display screen displayed by a terminal apparatus included in the information processing system of the first embodiment;

FIG. 7 is a flowchart illustrating an example of an autonomic nervous indicator measurement process of the first embodiment;

FIG. 8 is a diagram illustrating another conceptual example of the display screen displayed by the terminal apparatus of the first embodiment;

FIGS. 9A and 9B are flowcharts illustrating examples of an individual report output process of the first embodiment;

FIG. 10 is a table illustrating a conceptual example of an improvement plan database of the first embodiment;

FIG. 11 is a diagram illustrating an example of an individual report of the first embodiment;

FIGS. 12A and 12B are diagrams illustrating examples of a comparison screen of the first embodiment;

FIGS. 13A and 13B are flowcharts illustrating examples of an organization report output process of the first embodiment;

FIGS. 14 to 17 are diagrams illustrating examples of an organization report of the first embodiment;

FIG. 18 is a diagram illustrating an exemplary functional configuration of an information processing apparatus according to a second embodiment of the present invention;

FIGS. 19A and 19B are flowcharts illustrating exemplary processes of the information processing apparatus of the second embodiment;

FIG. 20 is a diagram illustrating an exemplary functional configuration of an information processing apparatus according to a third embodiment of the present invention;

FIG. 21 is a flowchart illustrating an example of an individual report output process of the third embodiment; and

FIG. 22 is a flowchart illustrating an example of an organization report output process of the third embodiment.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described in detail below. As used herein, the singular forms “a,” “an,” and “the” are intended to include the plural forms as well, unless the context clearly indicates otherwise.

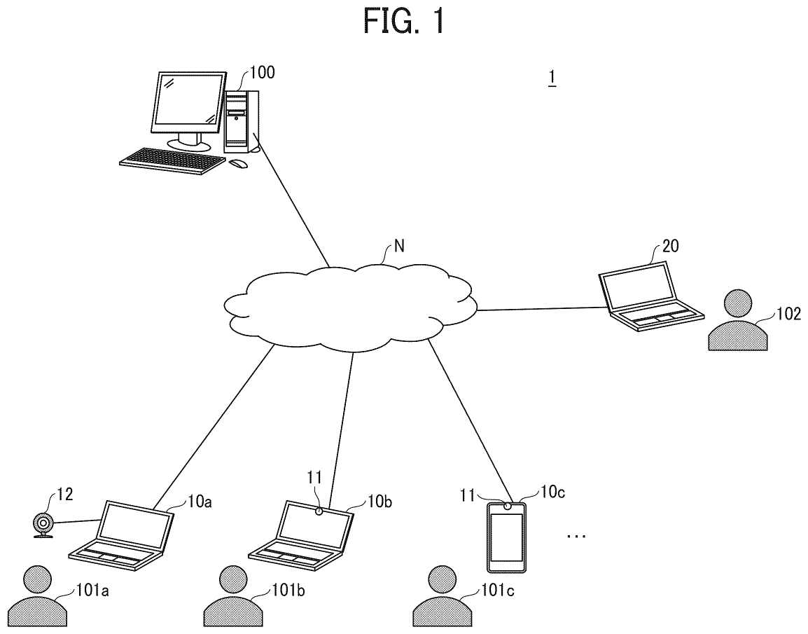

FIG. 1 is a diagram illustrating an exemplary system configuration of an information processing system 1 according to a first embodiment of the embodiment. The information processing system 1 includes a plurality of terminal apparatuses 10a, 10b, 10c, and so forth, an information processing apparatus 100, and an administrator terminal 20, which are connected to a communication network N such as the Internet or a local area network (LAN), for example. In the following description, any of the terminal apparatuses 10a, 10b, 10c, and so forth will be referred to as the terminal apparatus 10.

The terminal apparatus 10 is a computer, such as a personal computer (PC), a tablet terminal, or a smartphone, used by a user 101a, 101b, 101c, or the like in an environment to be evaluated. In the following description, any of the users 101a, 101b, 101c, and so forth will be referred to as the user 101.

As a specific example, the user 101 is an employee of a company, and the terminal apparatus 10 is a computer used for work by the employee. Herein, the company is an example of an organization, and the employee is an example of a member of the organization.

The terminal apparatus 10 is configured to capture an image (e.g., video image) of the user 101 using the terminal apparatus 10 with an image capturing device 11 built in the terminal apparatus 10 or an image capturing device 12 connected to the terminal apparatus 10, for example.

The information processing apparatus 100 is an information processing apparatus configured as a computer or a system including a plurality of computers, for example. With the terminal apparatus 10, the information processing apparatus 100 acquires motivation indicators and condition indicators. The motivation indicators are information indicating the motivation of the user 101 (an example of first information). The condition indicators are information indicating conditions related to the health of the user 101 (an example of second information). The information processing apparatus 100 further has a function of predicting and outputting indicators associated with the organization based on the acquired information.

As a specific example, the users 101 are employees of a company, and the information processing apparatus 100 provides a health management support service, which provides information related to health management indicators managed by the company based on the motivation indicators and the condition indicators of the employees.

Herein, the health management indicators are indicators managed by the company for health management, and the health management is viewing the health management of the employees from a managerial standpoint and strategically practicing the health management.

For example, by practicing the health management with the health management indicators, the company may be accredited as a corporate with excellent health and productivity management by the Ministry of Economy, Trade and Industry (METI) of Japan, or may receive the Excellence in Corporate Health Achievement Award (eCHAA) by the American College of Occupational and Environmental Medicine (ACOEM) in the United States.

The health management indicators include work engagement, engagement of employees, presenteeism, absenteeism, turnover rate, temporary leave rate, work performance evaluation, stress test score, subjective sense of health, degree of work satisfaction, health risk indicators including body mass indicator (BMI), blood pressure, blood sugar level, and lipid level, follow-up checkup rate, well-being indicator, and number of potentially serious cases, for example. The health management indicators may be any indicators managed by the company for the health management. For example, the health management indicators may be indicators described in the guidelines for health investment management accounting set by the METI, such as indicators related to the progress of health investment measures, indicators related to changes in awareness and behavior of employees, and ultimate goal indicators for health-related issues, or may be other indicators recommended by the METI or ACOEM. The information processing apparatus 100 may output one or more health management indicators.

The motivation indicators are indicators obtained by quantifying the result of evaluating, by questionnaire, factors included in the motivator factors and the hygiene factors of Herzberg's two-factor theory, for example. The motivator factors are factors that bring satisfaction in work, such as work fulfilment, sense of achievement in work, recognition for achievement, recognition from supervisors and colleagues, growth opportunities, and responsibility, for example. The motivator factors may include any other factor that brings satisfaction in work.

The hygiene factors are factors that bring dissatisfaction in work, such as company policies and management, working conditions, interpersonal relations, supervision, status, fair payment, job security, human resource system, and private life, for example. The hygiene factors may include any other factor that brings dissatisfaction in work.

The condition indicators are indicators indicating conditions of the employee (i.e., the user 101). In the first embodiment, the condition indicators include autonomic nervous indicators. Preferably, the information processing system 1 acquires the autonomic nervous indicators with the image capturing device 11 or 12 built in or connected to the terminal apparatus 10 used for work by the employee (the user 101).

The autonomic nervous indicators are indicators calculated by analyzing fluctuations in pulse wave or heart rate, for example, such as low frequency/high frequency (LF/HF), total power (TP), low frequency (LF), high frequency (HF), very low frequency (VLF), heart rate (HR), standard deviation of normal-to-normal interval (SDNN), and coefficient of variation of the R-R interval (CVRR). The autonomic nervous indicators are objective indicators not causing issues occurring in typical questionnaire-based subjective evaluation, thereby enabling more accurate evaluation of the conditions of the user 101. The issues occurring in the subjective evaluation include that a subject is not correctly aware of the conditions and that a subject is correctly aware of the conditions but does not provide honest answers, for example.

The inventors of the present application have found by examination that the autonomic nervous indicators affect each of the health management indicators. With the autonomic nervous indicators used as the condition indicators, therefore, the health management indicators are accurately predicted with less parameters. Further, the autonomic nervous indicators are acquired with the image capturing device 11 or 12 built in or connected to the terminal apparatus 10 used for work by the user 101, enabling a survey to be carried out regardless of the work location of the user 101.

For example, the information processing system 1 of the first embodiment is capable of conducting the survey even when the employee (the user 101) is working remotely from home or on a business trip, for example. The information processing system 1 is therefore able to obtain the condition indicators of the employee (the user 101) in real time.

The condition indicators other than the autonomic nervous indicators may include vital data such as respiration rate, oxygen saturation, blood pressure, body weight, and body composition and subjective data such as feeling of fatigue, feeling of stress, feeling of sleepiness, and feeling of not being adequately fed, for example.

The administrator terminal 20 is a computer used by an administrator 102, such as a PC, a tablet terminal, or a smartphone, for example. With the administrator terminal 20, the administrator 102 obtains the indicators predicted by the information processing apparatus 100 and the motivation indicators and the condition indicators of the user 101 acquired by the information processing apparatus 100, for example.

As a specific example, the administrator terminal 20 is a terminal apparatus used for work by an administrator user of a company that has introduced the health management support service provided by the information processing apparatus 100. The administrator user may be a person in a managerial position or in the human resource department of the company, or may be a member of occupational health personnel belonging to the company or contracting with the company such as an occupational physician or a public health nurse, for example.

The administrator terminal 20 may be or may not be equipped with an image capturing device. The administrator user is able to use an administrator function of the health management support service with a world wide web (Web) browser or an application compatible with the information processing system 1, for example, at his or her work location (e.g., office, home, or any other place).

According to the related-art technique, the health management indicators are obtained by census survey or pulse survey, for example. The census survey includes many questions, which increases the accuracy of the survey. The census survey, however, takes places infrequently (e.g., once a year). Meanwhile, the pulse survey includes less questions and thus may be carried out more frequently (e.g., once a week or month). The pulse survey, however, obtains subjective evaluations alone, which reduces the accuracy of the survey.

According to the information processing apparatus 100 of the first embodiment, the health management indicators are calculated with the motivation indicators of the user 101 and the condition indicators including the autonomic nervous indicators measured with the terminal apparatus 10 used by the user 101. As described above, it has been found that the autonomic nervous indicators are objective indicators affecting each of the health management indicators. With the autonomic nervous indicators, therefore, the information processing apparatus 100 predicts the health management indicators with high accuracy. Further, the information processing apparatus 100 acquires the autonomic nervous indicators with the terminal apparatus 10 used by the user 101, and thus is able to frequently conduct the survey to acquire the autonomic nervous indicators. According to the information processing apparatus 100 of the first embodiment, therefore, the health management indicators managed by the company are obtained accurately and frequently.

The health management indicators managed by the company are an example of the indicators associated with the organization. For example, in various organizations other than the company, the information processing apparatus 100 may predict various indicators associated with the organizations. Further, the survey is an example of an inquiry. The inquiry may include questionnaire and vote, for example, in addition to the survey.

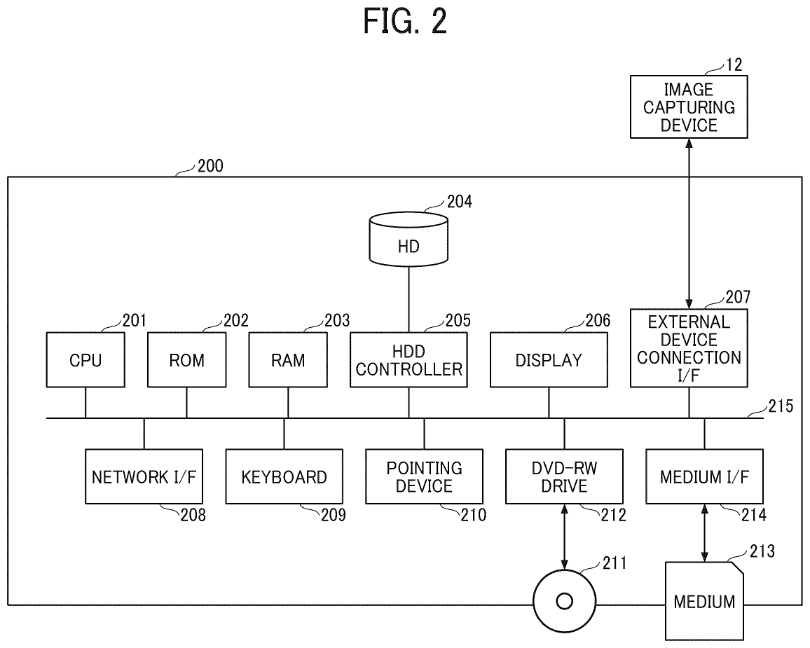

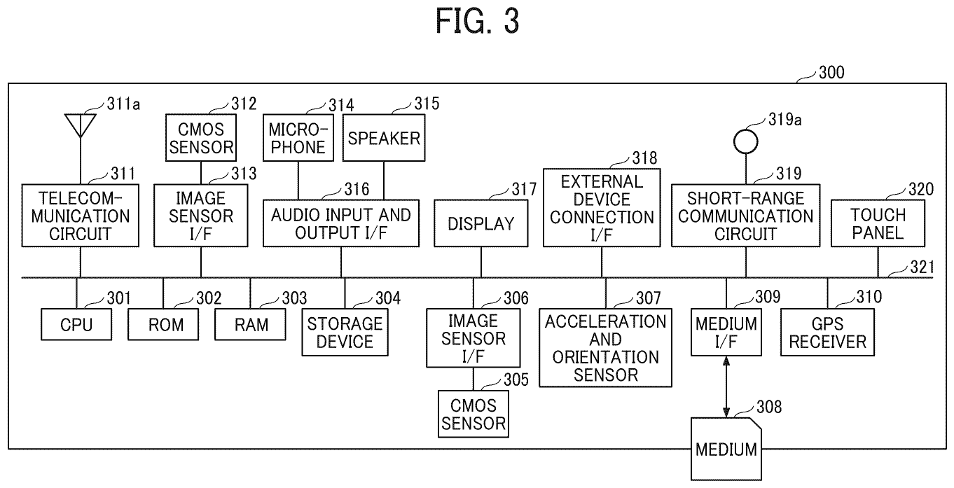

Each of the terminal apparatus 10, the administrator terminal 20, and the information processing apparatus 100 has the hardware configuration of a computer 200 illustrated in FIG. 2, for example. The information processing apparatus 100 may be implemented by a plurality of computers 200. The terminal apparatus 10 or the administrator terminal 20 may has the hardware configuration of an information terminal 300 described later with FIG. 3.

FIG. 2 is a diagram illustrating an exemplary hardware configuration of the computer 200 of the first embodiment. As illustrated in FIG. 2, the computer 200 includes a central processing unit (CPU) 201, a read only memory (ROM) 202, a random access memory (RAM) 203, a hard disk (HD) 204, a hard disk drive (HDD) controller 205, a display 206, an external device connection interface (I/F) 207, a network I/F 208, a keyboard 209, a pointing device 210, a digital versatile disk-rewritable (DVD-RW) drive 212, a medium I/F 214, and a bus line 215, for example.

The CPU 201 controls overall operation of the computer 200. The ROM 202 stores programs and data used to start the computer 200, such as a boot file, for example. The RAM 203 is used as a work area of the CPU 201, for example.

The HD 204 stores an operating system (OS), programs such as applications and drivers, and various data, for example. The HDD controller 205 controls the writing and reading of various data to and from the HD 204 under the control of the CPU 201, for example. The HD 204 and the HDD controller 205 are an example of a storage device included in the computer 200.

The display 206 displays various information such as a cursor, menus, windows, text, and images, for example. The display 206 may be provided outside the computer 200. The external device connection I/F 207 is an interface for connecting an external device to the computer 200. Herein, the external device includes the image capturing device 12, for example. The computer 200 may include the image capturing device 11 as a built-in device.

The network I/F 208 is an interface for performing data communication via the communication network N, for example. The keyboard 209 is a type of input means with a plurality of keys for inputting text, numerical values, and various instructions, for example. The pointing device 210 is a type of input means for selecting and executing various instructions, selecting a processing target, and moving the cursor, for example. The keyboard 209 and the pointing device 210 may be provided outside the computer 200.

The DVD-RW drive 212 controls the writing and reading of various data to and from a DVD-RW 211 as an example of a removable recording medium. The DVD-RW 211 is not limited to the DVD-RW, and may be another type of removable recording medium. The medium I/F 214 controls the writing (i.e., storage) and reading of data to and from a medium 213 such as a flash memory. The bus line 215 includes address buses, data buses, and various control signal lines for electrically connecting the above-described components to each other.

FIG. 3 is a diagram illustrating an exemplary hardware configuration of the information terminal 300 of the first embodiment. The terminal apparatus 10 may have the hardware configuration of the information terminal 300 illustrated in FIG. 3, for example.

As illustrated in FIG. 3, the information terminal 300 includes a CPU 301, a ROM 302, a RAM 303, a storage device 304, a complementary metal oxide semiconductor (CMOS) sensor 305, an image sensor I/F 306, an acceleration and orientation sensor 307, a medium I/F 309, and a global positioning system (GPS) receiver 310.

The CPU 301 executes a particular program to control overall operation of the information terminal 300. The ROM 302 stores a program used to drive the CPU 301, such as an initial program loader (IPL), for example. The RAM 303 is used as a work area of the CPU 301, for example. The storage device 304 is a high-capacity storage device that stores an OS, programs such as applications, and various data, for example.

The CMOS sensor 305 is a type of built-in image capturing means for capturing the image of a subject (mainly a user) under the control of the CPU 301 to obtain image data. The information terminal 300 may include a different type of image capturing means such as a charge coupled device (CCD) sensor in place of the CMOS sensor 305. The image sensor I/F 306 is a circuit that controls the driving of the CMOS sensor 305. The CMOS sensor 305 and the image sensor I/F 306 are an example of the image capturing device 11 built in the terminal apparatus 10.

The acceleration and orientation sensor 307 includes various sensors such as an electromagnetic compass that detects geomagnetism, a gyrocompass, and an acceleration sensor. The medium I/F 309 controls the writing (i.e., storage) and reading of data to and from a medium (i.e., storage medium) 308 such as a flash memory. The GPS receiver 310 is a positioning device that receives GPS signals (i.e., positioning signals) from GPS satellites.

The information terminal 300 further includes a telecommunication circuit 311, an antenna 311a for the telecommunication circuit 311, a CMOS sensor 312, an image sensor I/F 313, a microphone 314, a speaker 315, an audio input and output I/F 316, a display 317, an external device connection I/F 318, a short-range communication circuit 319, an antenna 319a for the short-range communication circuit 319, and a touch panel 320, for example.

The telecommunication circuit 311 is a circuit that communicates with another device or apparatus via the communication network N, for example. The CMOS sensor 312 is a type of built-in image capturing means for capturing the image of a subject under the control of the CPU 301 to obtain image data. The image sensor I/F 313 is a circuit that controls the driving of the CMOS sensor 312. The microphone 314 is a built-in device that converts sound into electrical signal. The speaker 315 is a built-in device that converts electrical signal into physical vibration to produce the sound of music or voice, for example.

The audio input and output I/F 316 is a circuit that processes the input of acoustic wave signal from the microphone 314 and the output of acoustic wave signal to the speaker 315 under the control of the CPU 301.

The display 317 is a type of display means, such as a liquid crystal or organic electroluminescence (EL) display, that displays the image of the subject and various icons, for example. The external device connection I/F 318 is an interface for connecting various external devices to the information terminal 300. The short-range communication circuit 319 includes a circuit that performs short-range wireless communication. The touch panel 320 is a type of input means for the user to operate the information terminal 300 by pressing the display 317.

The information terminal 300 further includes a bus line 321. The bus line 321 includes address buses, data buses, and various control signal lines for electrically connecting the CPU 301 and the other components of FIG. 3 to each other.

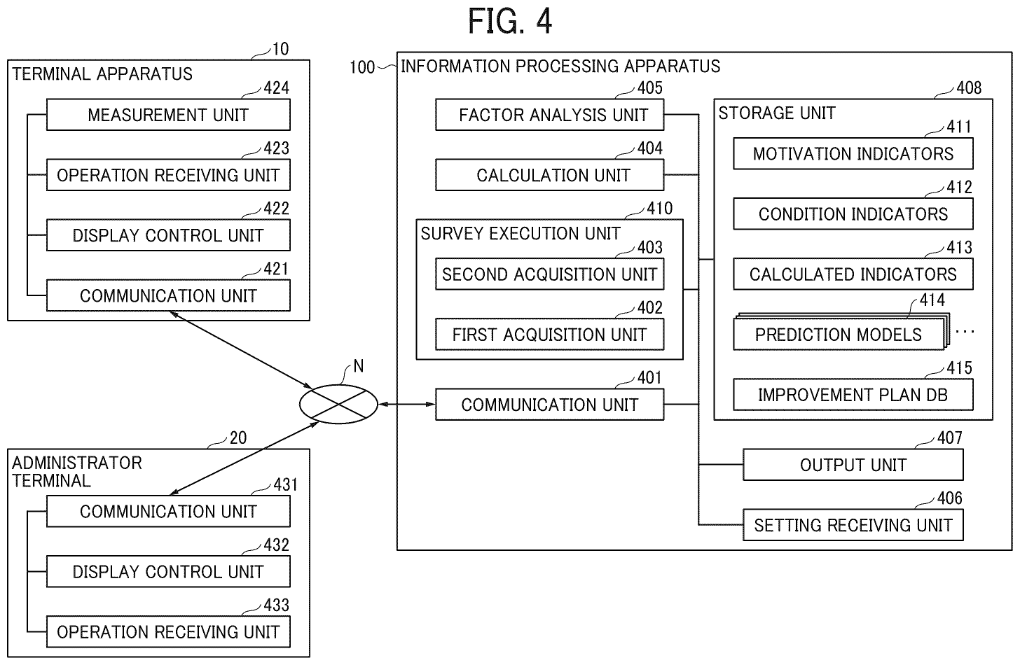

FIG. 4 is a diagram illustrating an exemplary functional configuration of the information processing system 1 of the first embodiment.

A description will first be given of a functional configuration of the information processing apparatus 100.

In the information processing apparatus 100, the CPU 201 executes a particular program stored in a storage medium such as the HD 204, for example, to implement functional units such as a communication unit 401, a first acquisition unit 402, a second acquisition unit 403, a calculation unit 404, a factor analysis unit 405, a setting receiving unit 406, and an output unit 407. At least part of these functional units may be implemented by hardware.

Further, in the information processing apparatus 100, a program executed by the CPU 201, the HD 204, and the HDD controller 205, for example, implement a storage unit 408.

The communication unit 401 performs a communication process of connecting the information processing apparatus 100 to the communication network N with the network I/F 208, for example, and communicating with another apparatus such as the terminal apparatus 10 or the administrator terminal 20.

The first acquisition unit 402 performs a first acquisition process of acquiring the motivation indicators (i.e., information indicating motivation) of the user 101 from an answer to an inquiry to the user 101 input to the terminal apparatus 10. For example, the first acquisition unit 402 causes the terminal apparatus 10 to display a subjective evaluation screen for asking simple questions that may be answered by the user 101 in a short time, such as the questions of a pulse survey. Then, based on the answer provided by the user 101, the first acquisition unit 402 acquires the motivation indicators of the user 101. The first acquisition unit 402 further adds identification information for identifying the user 101 to the acquired motivation indicators of the user 101, and stores and manages the motivation indicators of the user 101 in the storage unit 408, for example, chronologically and for the individual user 101.

The second acquisition unit 403 performs a second acquisition process of acquiring the condition indicators, i.e., the information indicating the conditions related to the health of the user 101, with the terminal apparatus 10 from the above-described inquiry to the user 101. For example, the second acquisition unit 403 acquires the autonomic nervous indicators of the user 101 measured based on the captured image of the user 101 captured with the image capturing device 11 built in the terminal apparatus 10 or the image capturing device 12 connected to the terminal apparatus 10. The second acquisition unit 403 further adds identification information for identifying the user 101 to the acquired condition indicators including the autonomic nervous indicators as information indicating the activity of the autonomic nervous system of the user 101, and stores and manages the condition indicators of the user 101 in the storage unit 408, for example, chronologically and for the individual user 101. A specific example of the process of acquiring the autonomic nervous indicators will be described later.

The second acquisition unit 403 may cause the terminal apparatus 10 to display the subjective evaluation screen and acquire the condition indicators of the user 101 (i.e.., the information indicating the conditions related to the health of the user 101) based on an answer provided by the user 101. The condition indicators of the user 101 may thus be subjective information related to the health of the user 101 or objective information related to the health of the user 101. Further, the condition indicators related to the health of the user 101 may be a combination of the objective information related to the health of the user 101 and the subjective information related to the health of the user 101.

The functions of the first acquisition unit 402 and the second acquisition unit 403 may be implemented by a single survey execution unit 410, as illustrated in FIG. 4, for example.

The calculation unit 404 performs a calculation process of calculating the indicators associated with the organization based on the motivation indicators of the user 101 and the condition indicators related to the health of the user 101.

As a specific example, the calculation unit 404 calculates the health management indicators by using prediction models created with the motivation indicators and the condition indicators of the members of the organization as explanatory variables and the health management indicators managed by the organization as objective variables. The health management indicators managed by the organization are an example of the indicators associated with the organization. The calculation unit 404 does not necessarily use the prediction models to calculate the indicators associated with the organization. For example, the calculation unit 404 may calculate the indicators associated with the organization based on particular evaluation equations, for example.

The storage unit 408 stores a plurality of prediction models 414 for predicting the indicators associated with the organization. With the plurality of prediction models 414, the calculation unit 404 calculates a plurality of indicators. Preferably, the plurality of prediction models 414 include one or more health management indicator prediction models for predicting the health management indicators.

The health management indicator prediction models are models for predicting the health management indicators from the condition indicators and the motivation indicators. The health management indicator prediction models are previously created and stored in, for example, the storage unit 408 of the information processing apparatus 100. For instance, data sets each including the condition indicators, the motivation indicators, and the health management indicators are acquired for a plurality of subjects. Then, the health management indicator prediction models are created with the condition indicators and the motivation indicators as the explanatory variables and the health management indicators as the objective variables. The health management indicator prediction models may be created with a method such as multivariate analysis, Bayesian network, or machine learning, for example.

For instance, a commonly used method such as multiple regression analysis or logistics regression analysis may be employed as the multivariate analysis method. For example, parameters as the coefficients of the explanatory variables may be calculated with multiple regression analysis to create the health management indicator prediction models for predicting the health management indicators as the objective variables from the condition indicators and the motivation indicators as the explanatory variables.

Further, a commonly used method may be employed as the Bayesian network method to calculate the parameters of the prediction models and thereby create the health management indicator prediction models.

Further, a commonly used method may be employed as the machine learning method. For example, a method such as decision tree, random forest, eXtreme gradient boosting (XGBoost), support vector machine (SBM), or neural network may be employed to calculate the parameters of the prediction models and thereby create the health management indicator prediction models.

It is desirable to create a health management indicator prediction model for each health management indicator, and to use the health management indicator prediction model corresponding to the health management indicator to be predicted. The data of the condition indicators and the motivation indicators used as the explanatory variables may be limited to the most recent data, but may also include past data to increase the accuracy of prediction. For example, if there is data of the condition indicators and the motivation indicators in a particular period (e.g., one month or less), the data in the particular period may also be included in the explanatory variables. A feature value such as the mean of the data in the particular period or the amount of change in the data in the particular period may be used as the explanatory variable. Further, information other than the condition indicators and the motivation indicators may be added to the explanatory variables. For example, later-described work information of the user 101 may be added to the explanatory variables.

The calculation unit 404 adds the identification information for identifying the user 101 to the calculated indicators, and stores and manages the indicators in the storage unit 408, for example, chronologically and for the individual user 101.

The factor analysis unit 405 performs a factor analysis process of analyzing factors highly influential on the indicators associated with the organization. As a specific example, the factor analysis unit 405 analyzes factors affecting the health management indicators and determines the order of priority of factors to be addressed to improve the health management indicators. The factors include decline in condition level, decline in motivation level, workload, type of work, overpressure, interpersonal relations, insufficient support from workplace, recognition and remuneration, work-life balance, and decline in psychological capital, for example, which are information inferable from the analysis of the explanatory variables for predicting the health management indicators. For example, the order of priority of the factors may be determined by calculating the respective deviation values of the explanatory variables and setting priorities such that the lower the deviation value is, the higher the priority of the corresponding explanatory variable is.

An explanatory variable with a low deviation value lowers the predicted value of the corresponding health management indicator. The explanatory variable with the low deviation value is likely to be improved by adopting measures employed by an organization in which the explanatory variable has a high deviation value. Therefore, the health management indicator is improved by taking measures to address the explanatory variable with the low deviation value. The information for determining the order of priority of the factors is not limited to the deviation value, and may be different information. For example, the information for determining the order of priority of the factors may be the degree of influence of each of the explanatory variables obtained in the creation of the health management prediction model, the estimated degree of improvement of the health management indicator when an explanatory variable with a lower-than-average value is improved up to an average value, a factor with a large amount of change, or the cost for improvement, for example.

The setting receiving unit 406 performs a setting receiving process of receiving various settings related to the information processing system 1. For example, the setting receiving unit 406 receives the setting of the health management indicators, the setting of the degrees of importance of the health management indicators, the setting of an individual report display method, and the setting of an organization report display method.

The output unit 407 performs an output process of outputting the indicators calculated by the calculation unit 404, for example. For instance, the output unit 407 transmits the individual report to the user 101 to the terminal apparatus 10 via the communication unit 401. The output unit 407 further outputs the organization report to the administrator 102 to the administrator terminal 20 via the communication unit 401.

Alternatively, the output unit 407 may cause a display unit, such as the display 206 of the information processing apparatus 100, to display the organization report to the administrator 102.

The individual report to the user 101 includes information such as the motivation indicators acquired by the first acquisition unit 402, the condition indicators acquired by the second acquisition unit 403, and the factors analyzed by the factor analysis unit 405, for example.

Preferably, the individual report includes information such as improvement plans for improving the factors analyzed by the factor analysis unit 405. For example, the output unit 407 may acquire the improvement plans for the factors analyzed by the factor analysis unit 405 from an improvement plan database (DB) 415, in which improvement plans for different factors are previously stored.

The individual report enables the user 101 to objectively understand his or her conditions and acquire information for improving his or her conditions and motivation, and thus take steps to improve the health management indicators.

The organization report to the administrator 102 includes information of the organization administered by the administrator 102, such as the motivation indicators acquired by the first acquisition unit 402, the condition indicators acquired by the second acquisition unit 403, and the indicators calculated by the calculation unit 404, for example. Preferably, the organization report further includes information such as the factors analyzed by the factor analysis unit 405 and the improvement plans for improving the factors.

The organization report enables the administrator 102 to understand the conditions and motivations of the members of the organization administered by the administrator 102 and acquire information for improving the conditions and motivations, and thus take steps to improve the health management indicators. The organization report further enables the company that has introduced the information processing system 1 to accurately and frequently obtain the health management indicators managed by the company.

The storage unit 408 stores various information and data including motivation indicators 411 and condition indicators 412 of the plurality of users 101, calculated indicators 413, the plurality of prediction models 414, and the improvement plan DB 415, for example.

A functional configuration of the terminal apparatus 10 will be described. The following description will be given on the assumption that the terminal apparatus 10 has the hardware configuration of the computer 200 described above with FIG. 2.

In the terminal apparatus 10, the CPU 201 executes a particular program stored in a storage medium such as the HD 204, for example, to implement functional units such as a communication unit 421, a display control unit 422, an operation receiving unit 423, and a measurement unit 424. At least part of these functional units may be implemented by hardware.

The communication unit 421 performs a communication process of connecting the terminal apparatus 10 to the communication network N with the network I/F 208, for example, and communicating with another apparatus such as the information processing apparatus 100.

The display control unit 422 performs a display control process of displaying, on a display unit such as the display 206, a display screen for displaying a subjective evaluation screen, a measurement screen, or an individual report provided by the information processing apparatus 100, for example. Alternatively, the display control unit 422 may display, on the display unit, a display screen for displaying a subjective evaluation screen or a measurement screen created by an application compatible with the information processing system 1 and executed by the terminal apparatus 10.

The operation receiving unit 423 performs an operation receiving process of receiving an operation performed by the user 101 on the display screen displayed on the display unit by the display control unit 422, for example.

The measurement unit 424 performs part or all of a measurement process of measuring the autonomic nervous indicators of the user 101 based on the captured image of the user 101.

The communication unit 421 transmits, to the information processing apparatus 100, answers provided by the user 101 on the subjective evaluation screen displayed by the display control unit 422 or the information indicating the motivation such as the motivation indicators based on the answers provided by the user 101, for example. The communication unit 421 further transmits, to the information processing apparatus 100, data related to the measurement by the measurement unit 424 of the autonomic nervous indicators of the user 101.

A functional configuration of the administrator terminal 20 will be described. The following description will be given on the assumption that the administrator terminal 20 has the hardware configuration of the computer 200 described above with FIG. 2.

In the administrator terminal 20, the CPU 201 executes a particular program stored in a storage medium such as the HD 204, for example, to implement functional units such as a communication unit 431, a display control unit 432, and an operation receiving unit 433. At least part of these functional units may be implemented by hardware.

The communication unit 431 performs a communication process of connecting the administrator terminal 20 to the communication network N with the network I/F 208, for example, and communicating with another apparatus such as the information processing apparatus 100.

The display control unit 432 performs a display control process of displaying, on a display unit such as the display 206, a display screen for displaying a result display screen provided by the information processing apparatus 100, for example.

The operation receiving unit 433 performs an operation receiving process of receiving an operation performed by the administrator 102 on the display screen displayed on the display unit by the display control unit 432, for example. The administrator terminal 20 may have a functional configuration similar to that of the terminal apparatus 10.

A description will be given of a processing procedure of an information processing method of the first embodiment.

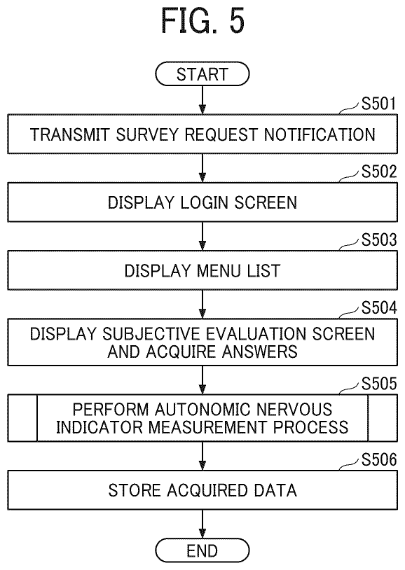

FIG. 5 is a flowchart illustrating an example of a survey process of the first embodiment. Specifically, FIG. 5 illustrates an example of a survey process in which the information processing system 1 acquires the motivation indicators and the condition indicators of the user 101.

At step S501, the survey execution unit 410 transmits a survey request notification to the terminal apparatus 10 of the user 101. As a method of transmitting the survey request notification, the survey execution unit 410 may transmit the survey request notification by electronic mail (email) or chat, for example. Preferably, the survey execution unit 410 transmits the survey request notification to an email address or chat service used for work by the user 101 to make the notification readily noticeable by the user 101.

The frequency of transmitting the survey request notification to the user 101 from the survey execution unit 410 is once a week, for example. The frequency of transmitting the survey request notification may be set as desired. To understand the state of the user 101 in more real-time, however, the frequency of transmitting the survey request notification is preferably at least once a month, and more preferably at least once a week.

The survey request notification may be transmitted on any day of the week. If it is intended to obtain the information of the normal state of the user 101, it is desirable to transmit the survey request notification after weekly days off (e.g., Monday if Saturday and Sunday are days off) before stress from working for consecutive days builds up. If it is intended to obtain the information of the stressed state of the user 101, on the other hand, it is desirable to transmit the survey request notification before weekly days off (e.g., Friday if Saturday and Sunday are days off).

Further, the survey request notification may be transmitted at any time. If it is intended to obtain the information of the normal state of the user 101, it is desirable to transmit the survey request notification before the start of the day's work, i.e.., before the stress from the day's work builds up. If it is intended to obtain the information of the stressed state of the user 101, on the other hand, it is desirable to transmit the survey request notification at a time near the end of the day's work.

The autonomic nervous indicators acquired from the survey vary depending on the day or time. It is therefore desirable to transmit the survey request notification at the same time on the same day of the week to allow the user 101 to take the survey almost at the same time on the same day of the week. It is also desirable that the administrator 102 or the user 101 is allowed to set the above-described method, frequency, day, and time of transmitting the survey request notification.

At step S502, the survey execution unit 410 causes the terminal apparatus 10 to display a login screen. For example, in response to receipt of the survey request notification, the user 101 accesses a particular uniform resource locator (URL) with a Web browser on the terminal apparatus 10. In response to the access, the survey execution unit 410 causes the terminal apparatus 10 to display the login screen for logging in to the service provided by the information processing apparatus 100.

The user 101 logs in to the service provided by the information processing apparatus 100 by inputting login information on the login screen displayed on the terminal apparatus 10, for example. However, any method may be used for the user 101 to log in to the service provided by the information processing apparatus 100. For example, the information processing apparatus 100 may be linked to another system used for work by the user 101 to allow the user 101 to log in to the service with login information of the user 101 for the another system, without need to input the login information.

Preferably, the survey execution unit 410 stores, in the storage unit 408, for example, basic information of the user 101 input by the user 101 when initially logging in to the service. The basic information includes information such as gender, age, date of birth, and mode of work (e.g., using remote work) of the user 101 and whether the user 101 has any underlying condition affecting the autonomic nervous function, for example.

At step S503, after the user 101 logs in to the service provided by the information processing apparatus 100, the survey execution unit 410 displays a menu list on the display screen displayed by the terminal apparatus 10. The menu list includes items such as “START SURVEY” for acquiring the condition indicators and the motivation indicators, “SURVEY RESULT” for displaying a survey result, and “DASHBOARD” for displaying the individual report or the organization report, for example. However, the items displayed in the menu list are not limited thereto. The user 101 starts the survey by selecting the item “START SURVEY” from the menu list, for example.

At step S504, the survey execution unit 410 displays the subjective evaluation screen on the display screen displayed by the terminal apparatus 10 and acquires the answers provided by the user 101.

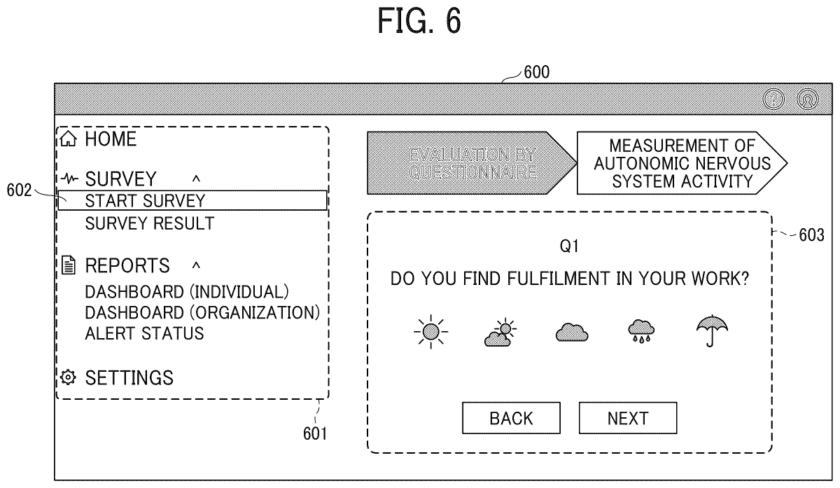

FIG. 6 is a diagram illustrating a conceptual example of the display screen displayed by the terminal apparatus 10 of the first embodiment. In the example of FIG. 6, a menu list 601 and a subjective evaluation screen 603 are displayed on a display screen 600 displayed by the terminal apparatus 10. The user 101 selects a “START SURVEY” item 602 from the menu list 601, for example, to start the survey.

The subjective evaluation screen 603 displays questions for evaluating the conditions and motivation of the user 101, for example. For instance, to evaluate the conditions of the user 101, the survey execution unit 410 displays questions such as “Are you in a good physical condition?” and “Are you sleeping well?” on the subjective evaluation screen 603. The questions may include any other question enabling the evaluation of a condition of the user 101.

Further, to evaluate the motivation of the user 101, the survey execution unit 410 displays questions such as “Do you think the current quantity and quality of your work are appropriate?” on the subjective evaluation screen 603 as questions related to the motivator factors. Further, the survey execution unit 410 displays questions such as “Do you have good relationships with your boss and colleagues?” on the subjective evaluation screen 603 as questions related to the hygiene factors. The questions may include any other question enabling the evaluation of the motivation of the user 101, but desirably include at least a question about the work fulfilment, the quantity and quality of work, the interpersonal relations in workplace, or the balance between work and private life, for example, which tends to change over time.

The survey execution unit 410 displays answer options to the questions on the subjective evaluation screen 603 and acquires answers selected by the user 101.

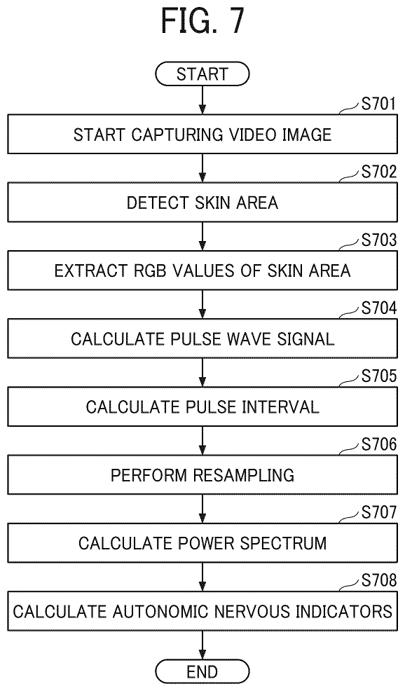

Referring back to step S505 in the flowchart of FIG. 5, the survey execution unit 410 performs an autonomic nervous indicator measurement process illustrated in FIG. 7, for example.

At step S506, the survey execution unit 410 stores the data acquired at steps S504 and S505 in the storage unit 408, for example.

FIG. 7 is a flowchart illustrating an example of the autonomic nervous indicator measurement process of the first embodiment. Specifically, FIG. 7 illustrates an example of an autonomic nervous indicator measurement process executed by the information processing system 1 at step S505 in FIG. 5.

At step S701, based on an instruction from the survey execution unit 410 of the information processing apparatus 100, the measurement unit 424 of the terminal apparatus 10 drives the image capturing device 11 or 12 to start capturing a video image of the user 101.

At step S702, the measurement unit 424 detects a skin area of the user 101 from the captured video image of the user 101 and extracts red, green, and blue (RGB) values of the detected skin area. For example, with a commonly used face recognition technique, the measurement unit 424 detects the coordinate positions of feature points (e.g., eyes, nose, and mouth) of the user 101 to detect the skin area of the face of the user 101 based on the detected coordinate positions. Herein, the skin area of the face desirably includes at least the skin area of checks. In the face, pulse wave signal is readily detected in the cheeks. By detecting the skin area of the face including the cheeks, therefore, the measurement unit 424 calculates the pulse wave signal more accurately.

The method of detecting the skin area may be any other method capable of detecting the skin area. For example, the measurement unit 424 may extract pixels with RGB values of typical skin color and detect a set of the extracted pixels as the skin area.

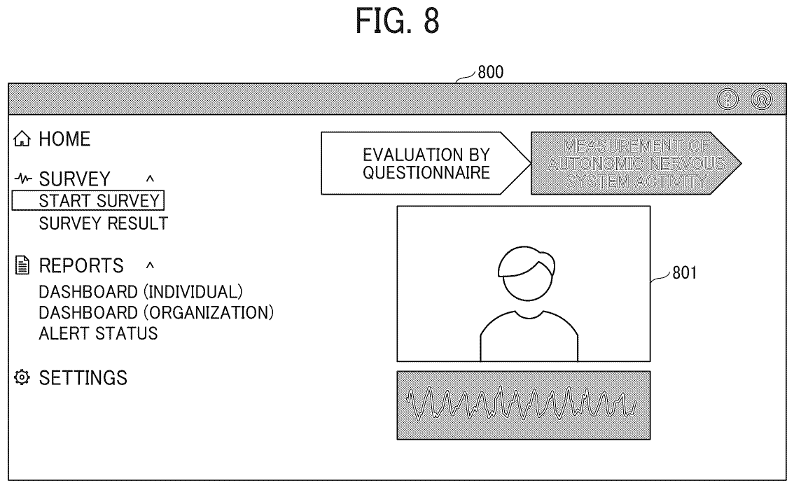

Further, as illustrated in FIG. 8, the measurement unit 424 may display a video image 801 of the user 101 on a display screen 800 displayed by the terminal apparatus 10 and identify the skin area based on a skin area specifying operation performed by the user 101, for example.

Further, the measurement unit 424 may detect the skin area as a plurality of divided areas, such as an area including the left check, an area including the right check, and an area including the nose, for example.

At step S703, the measurement unit 424 extracts the RGB values of the pixels included in the detected skin area. For example, the measurement unit 424 extracts, as the RGB values of the skin area, the values obtained by adding and averaging the R values, the G values, and the B values of the pixels included in the skin area. With the RGB values obtained by the adding and averaging process, the measurement unit 424 reduces the influence of noise on the pulse wave, thereby calculating the pulse wave signal more accurately.

The method of extracting the RGB values may be any other method capable of extracting the RGB values including the information of the pulse wave. For example, the measurement unit 424 may extract, as the RGB values of the skin area, the respective medians of the R values, the G values, and the B values of the pixels included in the skin area. The measurement unit 424 transmits the extracted RGB values to the information processing apparatus 100.

At step S704, the second acquisition unit 403 of the information processing apparatus 100 calculates the pulse wave signal from the RGB values of the skin area received from the terminal apparatus 10. For example, with a method of independent principal component analysis, the second acquisition unit 403 calculates the pulse wave signal, which represents a signal change component according to pulse, from chronological data of the RGB values of the skin area. The pulse wave signal represents chronological data in which the signal value changes periodically in accordance with the pulse. The method of calculating the pulse wave signal may be any other method capable of acquiring a pulse wave component. For example, since the pulse wave component is found most in the G value, the second acquisition unit 403 may use the G value as the pulse wave signal, or may use the value of the difference or ratio between the G value and the R or B value as the pulse wave signal.

At step S705, the second acquisition unit 403 calculates the pulse interval from the calculated pulse wave signal. The pulse interval may be calculated with an existing technique. For example, the second acquisition unit 403 may detect peak times of the pulse wave signal and calculate the pulse interval from the time interval between adjacent peaks.

At step S706, the second acquisition unit 403 performs resampling to equalize the time intervals of the chronological data of the calculated pulse interval. The time interval of the resampling is set to 0.25 seconds, for example. The signal value in the time lapsed in the resampling may be calculated by interpolation, for example. The method of interpolation may be linear or spline interpolation, for example.

At step S707, the second acquisition unit 403 calculates a power spectrum from the chronological data of the calculated pulse interval. As a method of calculating the power spectrum, a commonly used frequency analysis method may be used. For example, the second acquisition unit 403 may calculate the power at a specified frequency with the maximum entropy method. In the calculation of the power, parameters such as lag time are set to appropriate values.

At step S708, the second acquisition unit 403 calculates the autonomic nervous indicators based on the calculated power spectrum. The autonomic nervous indicators may include the LF/HF value indicating the balance of the autonomic nervous function and the TP value indicating overall activity of the autonomic nervous function, for example. The LF/HF value and the TP value may be calculated with a commonly used method. For example, the second acquisition unit 403 may set the integral of power in a frequency band higher than 0.04 Hz and equal to or lower than 0.15 Hz as the LF value and the integral of power in a frequency band higher than 0.15 Hz and equal to or lower than 0.40 Hz as the HF value, calculate the ratio between the LF value and the HF value as the LF/HF value, and calculate the sum of the LF value and the HF value as the TP value.

The survey process and the autonomic nervous indicator measurement process described above with FIG. 5 and FIG. 7, respectively, are illustrative. For example, therefore, the terminal apparatus 10 may execute an application compatible with the information processing system 1 to thereby execute the survey process or the autonomic nervous indicator measurement process in the terminal apparatus 10 and transmit the result of the executed process to the information processing apparatus 100. In this case, the information processing apparatus 100 does not execute the survey process; the first acquisition unit 402 and the second acquisition unit 403 of the information processing apparatus 100 acquire the motivation indicators and the condition indicators from the terminal apparatus 10.

Alternatively, the terminal apparatus 10 may execute the application compatible with the information processing system 1 to measure pulse wave data and transmit the measured pulse wave data to the information processing apparatus 100. In this case, the second acquisition unit 403 of the information processing apparatus 100 performs a process of calculating the autonomic nervous indicators based on the pulse wave data received from the terminal apparatus 10.

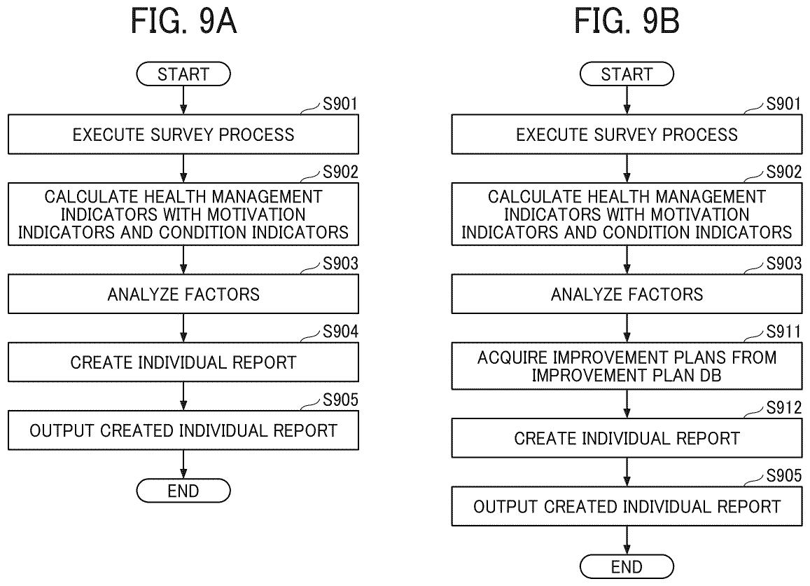

FIG. 9A is a flowchart illustrating an example of an individual report output process of the first embodiment. Specifically, FIG. 9A illustrates an example of a process in which the user 101 outputs the individual report with the terminal apparatus 10. The following description will be given on the assumption that a plurality of indicators indicating the performance of the user 101 and predicted by the information processing apparatus 100 are the health management indicators.

At step S901, the user 101 executes the survey process described above in steps S502 to S506 of FIG. 5 with the terminal apparatus 10. The user 101 may execute the survey process even without receipt of the survey request notification from the information processing apparatus 100.

At step S902, the calculation unit 404 calculates the health management indicators with the motivation indicators and the condition indicators of the user 101 stored in the storage unit 408, for example, in the survey process. For example, the calculation unit 404 inputs the motivation indicators and the condition indicators of the user 101 in a prediction model 414 for predicting a target health management indicator out of the plurality of prediction models 414 stored in the storage unit 408, to thereby calculate the health management indicator.

At step S903, the factor analysis unit 405 analyzes factors affecting the predicted health management indicators. For example, the factor analysis unit 405 analyzes the factors affecting the health management indicators and determines the order of priority of factors to be addressed to improve the health management indicators.

At step S904, the output unit 407 creates the individual report to be displayed by the terminal apparatus 10 used by the user 101. For example, the output unit 407 creates the individual report based on the acquired motivation indicators and condition indicators, the predicted health management indicators, and the analyzed factors.

At step S905, the output unit 407 outputs the created individual report to the terminal apparatus 10.

FIG. 9B is a flowchart illustrating another example of the individual report output process of the first embodiment. Specifically, FIG. 9B illustrates another example of the process in which the user 101 outputs the individual report with the terminal apparatus 10. The processes of steps S901 to S903 and S905 in the individual report output process of FIG. 9B are similar to those of FIG. 9A described above, and thus description thereof will be omitted here.

At step S911, the factor analysis unit 405 acquires improvement plans for the factors analyzed in step S903 from the improvement plan DB 415 stored in the storage unit 408, for example.

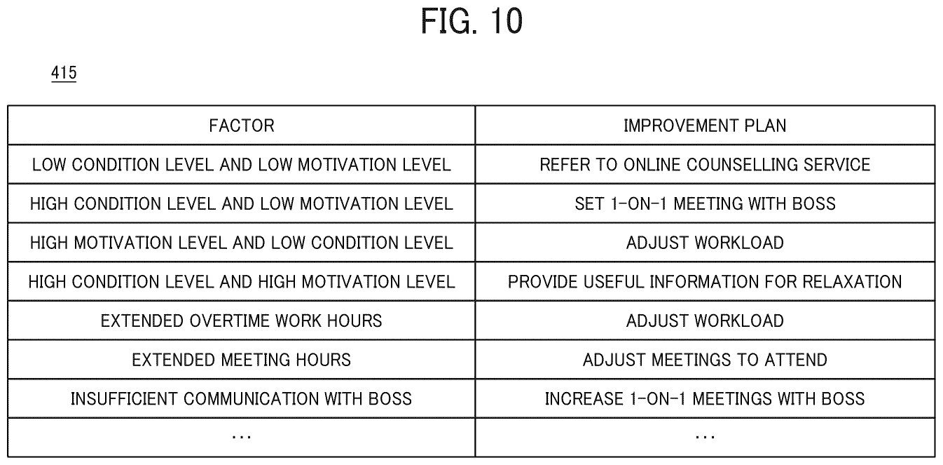

FIG. 10 is a table illustrating a conceptual example of the improvement plan DB 415 of the first embodiment. In the improvement plan DB 415, a factor and an improvement plan are stored in association with each other. For example, the factor is a factor or a combination of a plurality of factors affecting a health management indicator with a poor prediction result. The improvement plan is an improvement plan for the factor or the combination of a plurality of factors. The improvement plan may be providing or referring to related information or service, such as the referral to an occupational physician, the referral to a specialist, work visualization, manager training, team building training, 1-on-1 support, coating, psychological capital guiding training, employee assistance program (EAP), fitness, sleep support, knowledge sharing, communication creation and promotion, whistleblowing hotline, or resilience training, for example.

At step S912, the output unit 407 creates the individual report to be displayed by the terminal apparatus 10 used by the user 101. For example, the output unit 407 creates the individual report based on the acquired motivation indicators and condition indicators, the predicted health management indicators, the analyzed factors, and the acquired improvement plans.

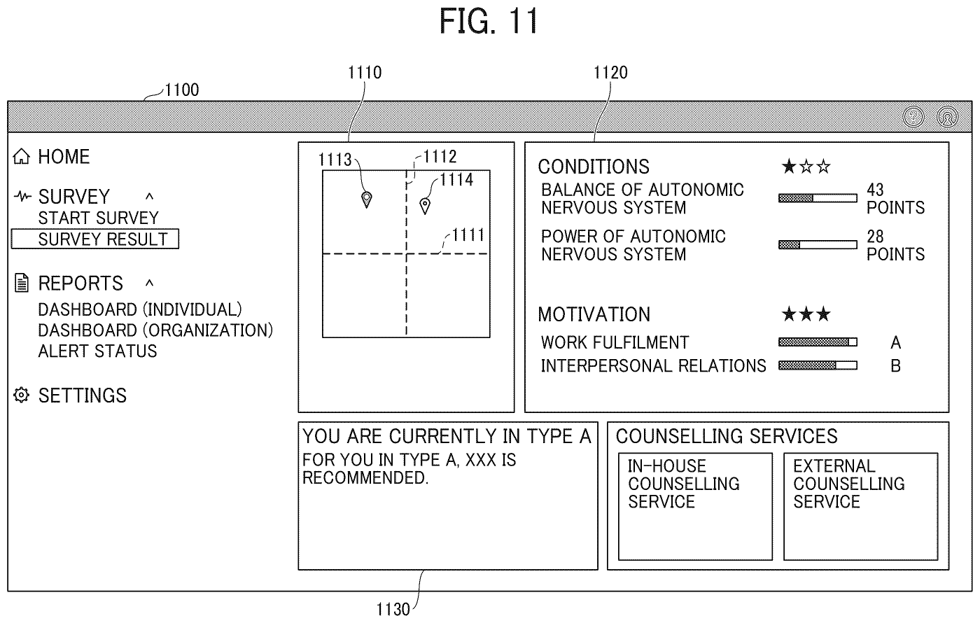

FIG. 11 is a diagram illustrating an example of the individual report of the first embodiment. FIG. 11 illustrates a conceptual example of the individual report created by the output unit 407 in step S912 of FIG. 9B.

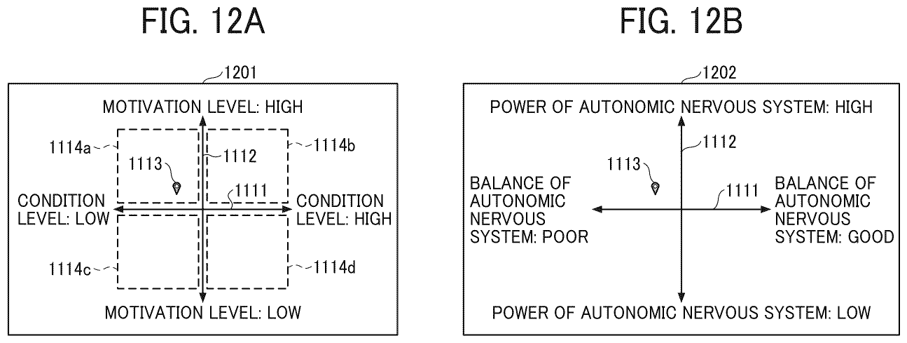

In the example of FIG. 11, an individual report 1100 includes a graph section 1110, a numerical value section 1120, and a factor section 1130, for example. The graph section 1110 is an area for displaying an analysis result as a graph. For instance, as illustrated in FIG. 12A, the output unit 407 displays a graph 1201 in the graph section 1110. In the graph 1201, the horizontal axis represents the numerical value of the condition indicators, and the vertical axis represents the numerical value of the motivation indicators. For example, the output unit 407 sets a center line 1111 along the horizontal axis as a standard condition value for evaluating the condition indicators, and sets a center line 1112 along the vertical axis as a standard motivation value for evaluating the motivation indicators. The output unit 407 further displays a marker 1113 in the graph 1201 to indicate which one of four quadrants 1114a, 1114b, 1114c, and 1114d includes the current indicators of the user 101. As illustrated in FIG. 11, the output unit 407 may further display a marker 1114 to indicate which one of the four quadrants 1114a to 1114d includes previous indicators of the user 101.

As another example, the output unit 407 may display a graph 1202 in the graph section 1110, as illustrated in FIG. 12B. In the graph 1202, the center line 1111 along the horizontal axis represents a standard value of the LF/HF value as an autonomic nervous indicator indicating the balance of the autonomic nervous function, and the center line 1112 along the vertical axis represents a standard value of the TP value as an autonomic nervous indicator indicating the total power of the autonomic nervous function. Further, the output unit 407 may set a value related to an objective value of the condition indicators (e.g., the TP value) as the center line along the horizontal axis, and may set a value related to a subjective value of the condition indicators as the center line along the vertical axis. The graph displayed in the graph section 1110 may be switched by the user 101.

The numerical value section 1120 is an area for displaying the numerical values of the condition indicators and the motivation indicators, for example. The factor section 1130 is an area for displaying the factors affecting the health management indicators, for example. The factor section 1130 may further display the improvement plants for improving the factors affecting the health management indicators, for example.

Effective improvement plans vary depending on which one of the four quadrants 1114a to 1114d includes the marker 1113 representing the indicators of the user 101. It is therefore desirable that the respective improvement plans for the four quadrants 1114a to 1114d are stored in the improvement plan DB 415. For example, if the marker 1113 is located in the quadrant 1114a, in which the condition indicators are at a low level and the motivation indicators are at a high level, the user 101 is in a near-workaholic (work-addicted) state. In this state, the user 101 may exhibit short-term high performance but has a risk of burnout from fatigue accumulation if this state continues. If the marker 1113 is located in the quadrant 1114a, therefore, it is desirable to provide improvement plans such as providing the user 101 with information or service for keeping the user 101 from accumulating fatigue and providing a manager of the user 101 with information encouraging communication with the user 101 or adjustment of the workload of the user 101.

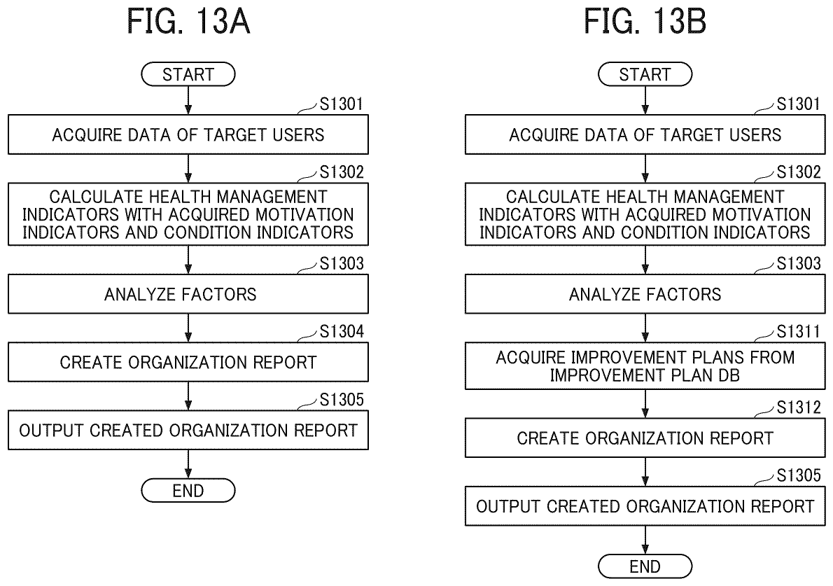

FIG. 13A is a flowchart illustrating an example of an organization report output process of the first embodiment. Specifically, FIG. 13A illustrates an example of a process in which the administrator 102 outputs the organization report with the administrator terminal 20. It is assumed here that, at the beginning of the organization report output process of FIG. 13A, the users 101a, 101b, 101c, and so forth, who are the members of the organization administered by the administrator 102, have executed the survey process described above with

FIG. 5, for example. The following description will be given on the assumption that a plurality of indicators indicating the performance of the user 101 and predicted by the information processing apparatus 100 are the health management indicators.

At step S1301, the calculation unit 404 acquires the data of the target users 101 for the organization report. For example, the calculation unit 404 acquires the motivation indicators and the condition indicators of the users 101a, 101b, 101c, and so forth, who are the members of the organization administered by the administrator 102, from the storage unit 408, for example.

At step S1302, the calculation unit 404 calculates the health management indicators with the acquired motivation indicators and condition indicators. For example, the calculation unit 404 inputs the motivation indicators and the condition indicators of the target users 101 in a prediction model 414 for predicting a target health management indicator out of the plurality of prediction models 414 stored in the storage unit 408, to thereby calculate the health management indicator.

At step S1303, the factor analysis unit 405 analyzes factors affecting the predicted health management indicators.

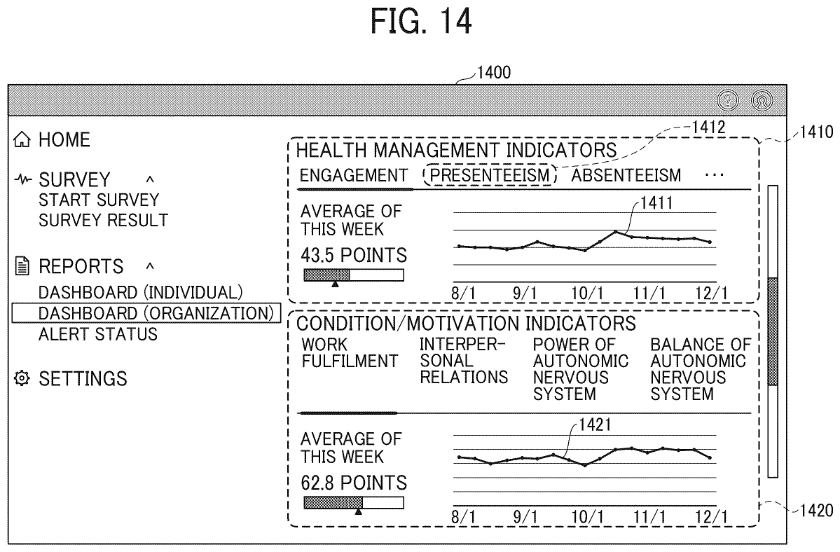

At step S1304, the output unit 407 creates the organization report to output to the administrator terminal 20. For example, the output unit 407 creates the organization report illustrated in FIGS. 14 to 17 with the acquired motivation indicators and condition indicators, the predicted health management indicators, and the analyzed factors.

At step S1305, the output unit 407 outputs the created organization report to the administrator terminal 20.

FIG. 13B is a flowchart illustrating another example of the organization report output process of the first embodiment. Specifically, FIG. 13B illustrates another example of the process in which the administrator 102 outputs the organization report with the administrator terminal 20. The processes of steps S1301 to S1303 and S1305 in the organization report output process of FIG. 13B are similar to those of FIG. 13A described above, and thus description thereof will be omitted here.

At step S1311, the factor analysis unit 405 acquires improvement plans corresponding to the analyzed factors from the improvement plan DB 415.

At step S1312, the output unit 407 creates the organization report to output to the administrator terminal 20. For example, the output unit 407 creates the organization report with the acquired motivation indicators and condition indicators, the predicted health management indicators, the analyzed factors, and the acquired improvement plans.

FIGS. 14 to 17 are diagrams illustrating examples of the organization report of the first embodiment. For instance, FIG. 14 illustrates an example of the organization report displayed by the administrator terminal 20 in accordance with the organization report output process of FIG. 13A. In the example of FIG. 14, an organization report 1400 includes a health management indicator display field 1410 and a condition and motivation indicator display field 1420.

The health management indicator display field 1410 displays, for example, a numerical value and a graph of each of health management indicators predicted by the information processing apparatus 100. The displayed value of the health management indicator may be a measure of central tendency (e.g., mean, median, minimum value, or maximum value) of the organization, or may be the value of a particular user 101.

In the example of FIG. 14, a graph 1411 is displayed in the health management indicator display field 1410. The graph 1411 represents changes in a health management indicator “ENGAGEMENT,” which is the health management indicator of the highest priority out of a plurality of health management indicators “ENGAGEMENT,” “PRESENTEEISM,” “ABSENTEEISM,” and so forth.

As described above, when there are a plurality of health management indicators, it is desirable that the output unit 407 displays the health management indicators of the highest priority in the health management indicator display field 1410. It is also desirable that the output unit 407 displays the health management indicators in descending order of priority in the health management indicator display field 1410. For example, in the health management indicator display field 1410 of FIG. 14, it is desirable that the higher the priority of the health management indicator is, the more left the tab of the health management indicator is located to make it easy for the administrator 102 to select the health management indicator.

The priorities of the health management indicators are determined based on the analysis of the health management indicators by the factor analysis unit 405, for example. The information processing apparatus 100 includes the setting receiving unit 406, which receives the respective degrees of importance of the health management indicators set by the administrator 102. The output unit 407 may preferentially output the health management indicator, the degree of importance of which set by the administrator 102 is higher than the other health management indicators. Herein, preferentially outputting the health management indicator refers to displaying the health management indicator of higher priority in the health management indicator display field 1410, for example, as described above.

The condition and motivation indicator display field 1420 displays, for example, a numerical value and a graph of each of condition indicators or motivation indicators acquired by the information processing apparatus 100.

In the example of FIG. 14, the condition and motivation indicator display field 1420 displays a graph 1421 of an indicator “WORK FULFILMENT,” which is causally related to the health management indicator “ENGAGEMENT” displayed in the health management indicator display field 1410. It is desirable that the output unit 407 thus displays, in the condition and motivation indicator display field 1420, the condition or motivation indicator causally related to the health management indicator displayed in the health management indicator display field 1410.

If there are a plurality of condition indicators or motivation indicators causally related to the health management indicator displayed in the health management indicator display field 1410, it is desirable that the output unit 407 displays the condition indicators or motivation indicators in descending order of priority.

For example, in the condition and motivation indicator display field 1420 of FIG. 14, it is desirable that the higher the priority of the condition or motivation indicator is, the more left the tab of the condition or motivation indicator is located to make it easy for the administrator 102 to select the condition or motivation indicator.

As a specific example, out of the condition or motivation indicators causally related to the health management indicator displayed in the health management indicator display field 1410, the output unit 407 may preferentially display a condition or motivation indicator, the value of which deviates from a standard value more than the other condition or motivation indicators.

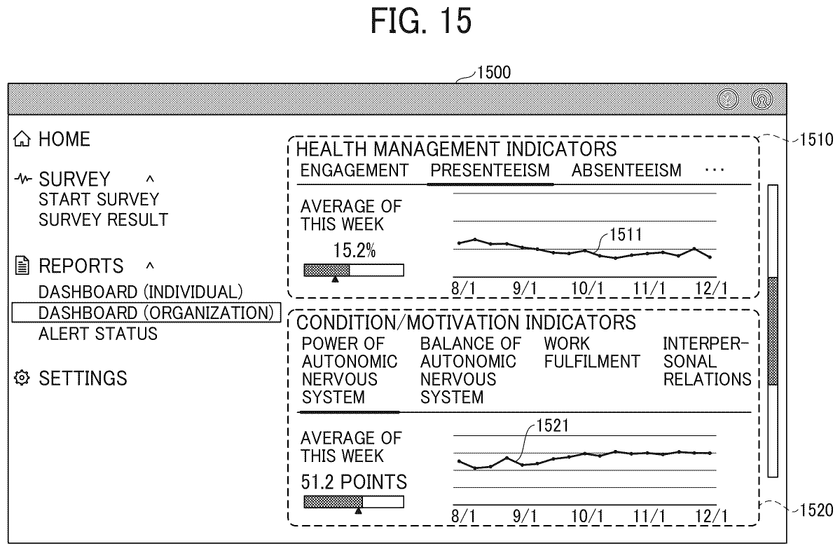

If a tab 1412 of the health management indicator “PRESENTEEISM” is selected in the health management indicator display field 1410, for example, the output unit 407 outputs an organization report 1500 illustrated in FIG. 15 to the administrator terminal 20.

It is desirable that the graph in the health management indicator display field 1410 and the graph in the condition and motivation indicator display field 1420 are displayed with the same display period or width to facilitate the administrator 102 to see the correspondence between the graphs.

FIG. 15 illustrates another example of the organization report displayed by the administrator terminal 20. In the example of FIG. 15, the organization report 1500 includes a health management indicator display field 1510 and a condition and motivation indicator display field 1520.

In the example of FIG. 15, the health management indicator display field 1510 displays a graph 1551 representing changes in the health management indicator “PRESENTEEISM” selected by the administrator 102 in place of the graph 1411 representing the changes in the health management indicator “ENGAGEMENT.” The output unit 407 thus selectively displays, in the health management indicator display field 1510, the graph or numerical value of the health management indicator selected by the administrator 102 out of the plurality of the health management indicators displayed in the health management indicator display field 1510.

Further, in the example of FIG. 15, the condition and motivation indicator display field 1520 displays a graph 1521 of an indicator “POWER OF AUTONOMIC NERVOUS SYSTEM,” which is causally related to the health management indicator “PRESENTEEISM” displayed in the health management indicator display field 1510. It is desirable that the output unit 407 thus changes the condition or motivation indicator displayed in the condition and motivation indicator display field 1520 in accordance with the health management indicator displayed in the health management indicator display field 1510.

The output unit 407 displays, in the condition and motivation indicator display field 1520, the graph or numerical value of the condition or motivation indicator selected by the administrator 102.

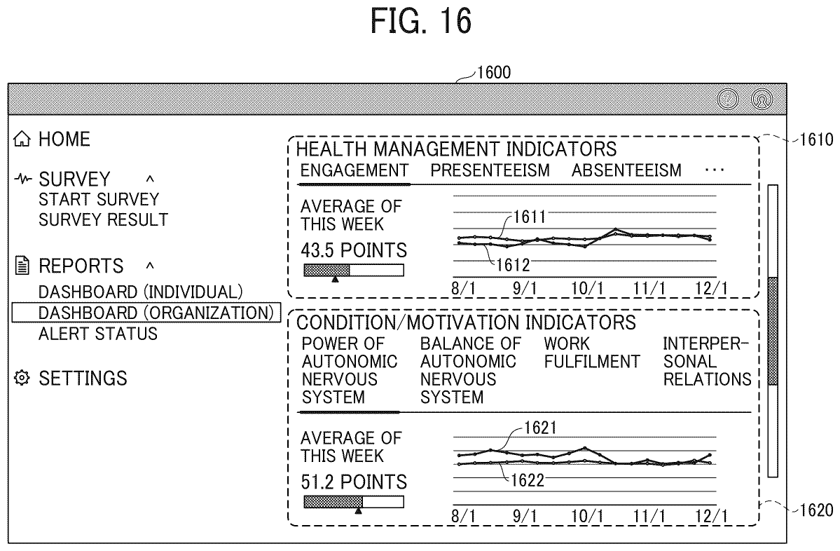

FIG. 16 illustrates another example of the organization report displayed by the administrator terminal 20. In the example of FIG. 16, an organization report 1600 includes a health management indicator display field 1610 and a condition and motivation indicator display field 1620.

In the example of FIG. 16, the health management indicator display field 1610 displays graphs representing changes in a plurality of health management indicators (e.g., a graph 1611 representing changes in the health management indicator “ENGAGEMENT” and a graph 1612 representing changes in the health management indicator “PRESENTEEISM”). The output unit 407 may thus display the graphs representing the changes in the plurality of health management indicators in the health management indicator display field 1610. For example, the output unit 407 may display, in the health management indicator display field 1610, graphs representing changes in a particular number of health management indicators of high priority or a particular number of health management indicators selected by the administrator 102.

Similarly, in the example of FIG. 16, the condition and motivation indicator display field 1620 displays a plurality of graphs representing changes in a plurality of condition or motivation indicators (e.g., a graph 1621 representing changes in the indicator “POWER OF AUTONOMIC NERVOUS SYSTEM” and a graph 1622 representing changes in the indicator “BALANCE OF AUTONOMIC NERVOUS SYSTEM”). The output unit 407 may thus display the plurality of graphs representing the changes in the condition or motivation indicators in the condition and motivation display field 1620. For example, the output unit 407 may display, in the condition and motivation indicator display field 1620, graphs representing changes in a particular number of condition or motivation indicators of high priority or a particular number of condition or motivation indicators selected by the administrator 102.

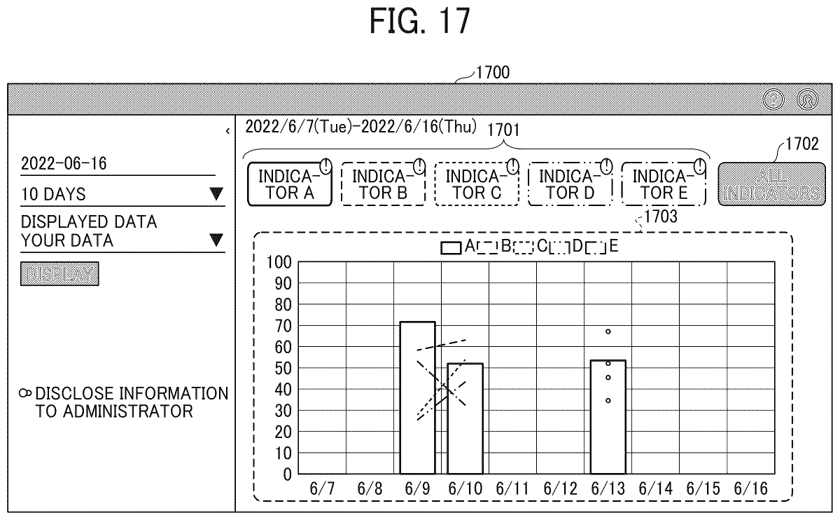

FIG. 17 illustrates another example of the organization report displayed by the administrator terminal 20. In the example of FIG. 17, an organization report 1700 includes selection buttons 1701 for selecting a plurality of indicators A, B, C, D, and E and a selection button 1702 for selecting all of the plurality of indicators A to E. Herein, the plurality of indicators A to E may be the health management indicators, the condition indicators, or the motivation indicators.

The output unit 407 displays, in a graph section 1703, graphs representing changes in a desired number of indicators selected with the selection buttons 1701 for selecting the plurality of indicators A to E, for example. If the selection button 1702 for selecting all of the indicators A to E is selected, the output unit 407 displays graphs representing changes in all of the indicators A to E in the graph section 1703. The output unit 404 may thus select all of the indicators A to E and output an organization report visualizing the selected indicators A to E.

A second embodiment of the present invention will be described.

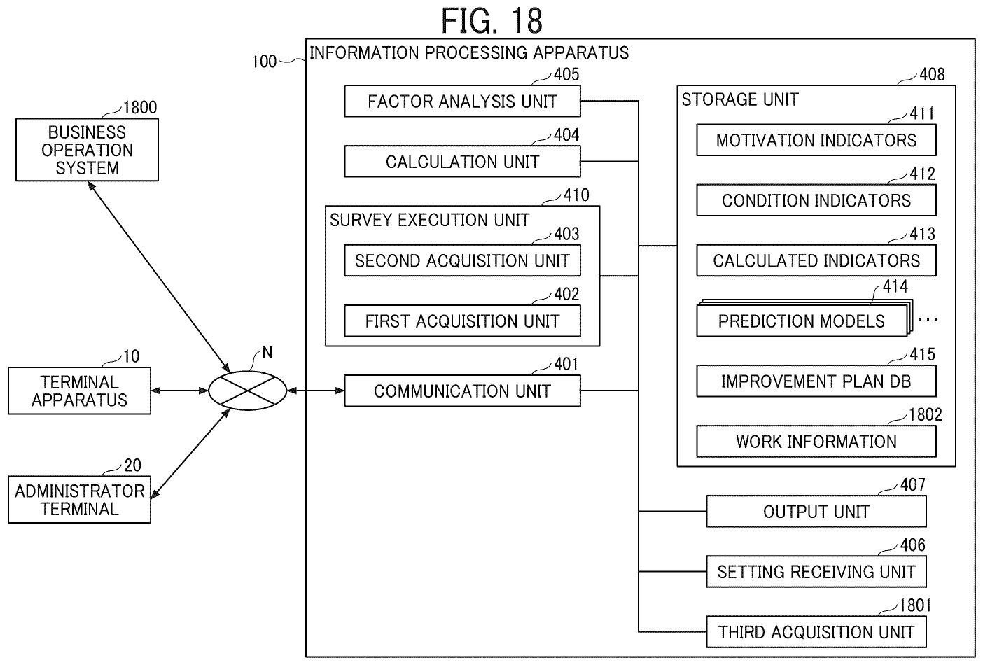

FIG. 18 is a diagram illustrating an exemplary functional configuration of the information processing apparatus 100 of the second embodiment. The information processing apparatus 100 of the second embodiment includes a third acquisition unit 1801 in addition to the functional units of the information processing apparatus 100 of the first embodiment described above with FIG. 4.

The third acquisition unit 1801 is implemented by the CPU 201 executing a particular program stored in a storage medium such as the HD 204, for example. The third acquisition unit 1801 acquires the work information of the user 101 from, for example, a business operation system 1800 that manages the work information of the user 101. The third acquisition unit 1801 adds the identification information for identifying the user 101 to the acquired work information of the user 101, and stores and manages the work information of the user 101 in the storage unit 408, for example, chronologically and for the individual user 101.

The business operation system 1800 is an external system used in work by the user 101, such as an attendance system, a schedule management system, an email system, a chat system, a meeting system, or a document management system, for example.

The work information acquired by the third acquisition unit 1801 includes at least one of the working hours, the overtime working hours, the hours of meetings, the number of meetings, the hours of 1-on-1 meetings with the boss, the number of 1-on-1 meetings with the boss, the number of email exchanges, the email usage time slots, the number of chat exchanges, the chat usage time slots, the time slots registered with schedules, the type of work, or the rest hours of the user 101, for example. The third acquisition unit 1801 acquires the work information from the business operation system 1800 during the execution of the survey process illustrated in FIG. 5 or with previously set particular timing (e.g., once a day or half a day), for example.

With the condition indicators, the motivation indicators, and the work information of the user 101, the calculation unit 404 of the second embodiment calculates a plurality of indicators indicating the performance of the user 101. As a specific example, the calculation unit 404 calculates the health management indicators of the user 101 with the prediction models 414 created with the condition indicators, the motivation indicators, and the work information of the members of the organization as the explanatory variables and the health management indicators managed by the organization as the objective variables. For example, for each of a plurality of subjects, the calculation unit 404 acquires a data set including the condition indicators, the motivation indicators, the work information, and the health management indicators. Then, the calculation unit 404 creates the prediction models 414 through analysis with a method such as multivariate analysis, Bayesian network, or machine learning with the condition indicators, the motivation indicators, and the work information as the explanatory variables and the health management indicators as the objective variables.

The factor analysis unit 405 of the second embodiment analyzes the factors affecting the health management indicators. The factor analysis unit 405 analyzes the factors based on information including the work information when determining the order of priority of the factors to be addressed to improve the health management indicators.

It is desirable that the output unit 407 of the second embodiment outputs the individual report or the organization report including the work information as a factor affecting the health management indicators.

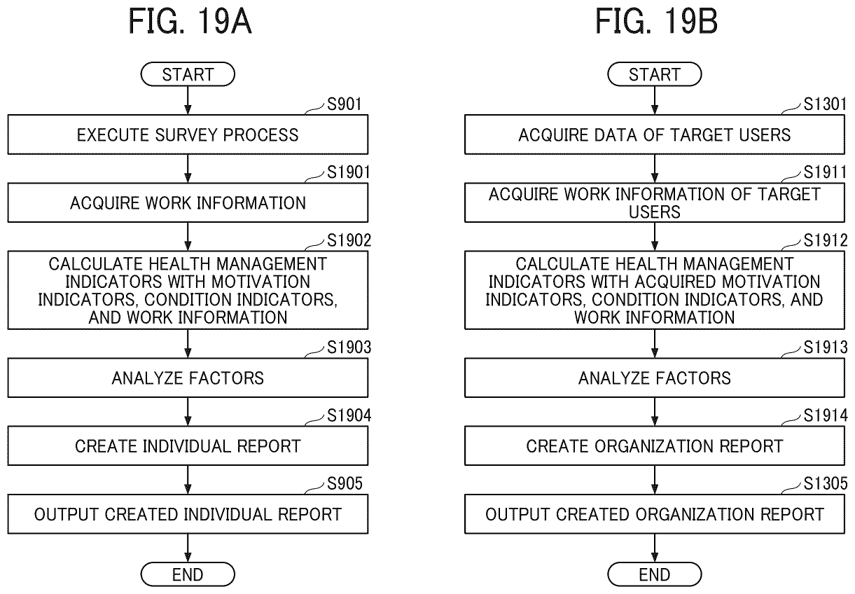

FIG. 19A illustrates an example of an individual report output process of the second embodiment. Specifically, FIG. 19A illustrates an example of an individual report output process performed by the information processing apparatus 100 of the second embodiment described above with FIG. 18. The processes of steps S901 and S905 in the individual report output process of FIG. 19A are similar to those of the individual report output process of the first embodiment described above with FIG. 9A, and thus description thereof will be omitted here. At step S1901, the third acquisition unit 1801 acquires the work information of the user 101. For instance, if the latest work information of the user 101 is already stored in the storage unit 408, for example, the third acquisition unit 1801 acquires the work information of the user 101 from the storage unit 408. If the latest work information of the user 101 is not stored in the storage unit 408, for example, the third acquisition unit 1801 acquires the work information of the user 101 from the business operation system 1800.

At step S1902, the calculation unit 404 calculates the health management indicators with the motivation indicators, the condition indicators, and the work information of the user 101. For example, the calculation unit 404 inputs the motivation indicators, the condition indicators, and the work information of the user 101 in a prediction model 414 for predicting a target health management indicator out of the plurality of prediction models 414 stored in the storage unit 408, to thereby calculate the health management indicator.

At step S1903, the factor analysis unit 405 analyzes factors affecting the predicted health management indicators. Preferably, the factor analysis unit 405 analyzes the factors affecting the health management indicators, including the work information.

At step S1904, the output unit 407 creates the individual report to be displayed by the terminal apparatus 10 used by the user 101. For example, the output unit 407 creates the individual report based on the acquired motivation indicators and condition indicators, the predicted health management indicators, and the analyzed factors, for example. Preferably, the output unit 407 creates the individual report including the work information as a factor affecting the health management indicators.

FIG. 19B illustrates an example of an organization report output process of the second embodiment. Specifically, FIG. 19B illustrates an example of an organization report output process performed by the information processing apparatus 100 of the second embodiment described above with FIG. 18. The processes of steps S1301 and S1305 in the organization report output process of FIG. 19B are similar to those of the organization report output process of the first embodiment described above with FIG. 13A, and thus description thereof will be omitted here.

At step S1911, the third acquisition unit 1801 acquires the work information of the target users 101. For example, the third acquisition unit 1801 acquires the work information of the users 101a, 101b, 101c, and so forth, who are the members of the organization administered by the administrator 102, from the business operation system 1800 or the storage unit 408, for example.

At step S1912, the calculation unit 404 calculates the health management indicators with the acquired motivation indicators, condition indicators, and work information of the target users 101. For example, the calculation unit 404 inputs the motivation indicators, the condition indicators, and the work information of the target users 101 in a prediction model 414 for predicting a target health management indicator out of the plurality of prediction models 414 stored in the storage unit 408, to thereby calculate the health management indicator.

At step S1903, the factor analysis unit 405 analyzes factors affecting the predicted health management indicators. Preferably, the factor analysis unit 405 analyzes the factors affecting the health management indicators, including the work information.

At step S1904, the output unit 407 creates the organization report to output to the administrator terminal 20. For example, the output unit 407 creates the organization report with the acquired motivation indicators, condition indicators, and work information, the predicted health management indicators, and the analyzed factors.

As described above, with the work information in addition to the motivation indicators and the condition indicators of the user 101, the information processing system 1 of the second embodiment predicts the indicators indicating the performance of the user 101.

A third embodiment of the present invention will be described.

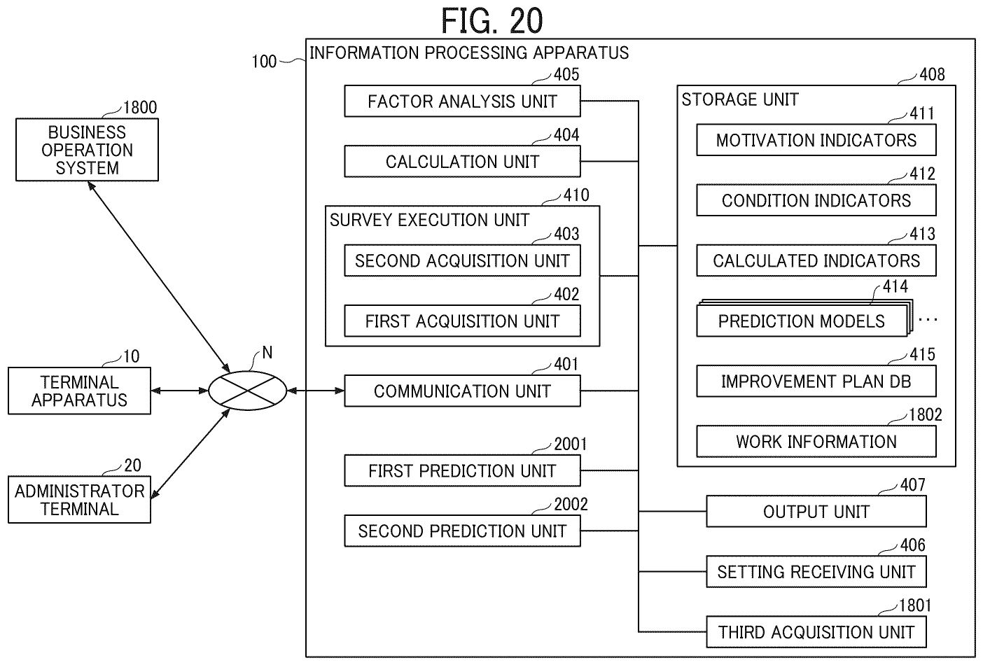

FIG. 20 is a diagram illustrating am exemplary functional configuration of the information processing apparatus 100 of the third embodiment. The information processing apparatus 100 of the third embodiment includes a first prediction unit 2001 and a second prediction unit 2002 in addition to the functional units of the information processing apparatus 100 of the second embodiment described above with FIG. 18. The first prediction unit 2001 and the second prediction unit 2002 are implemented by the CPU 201 executing a particular program stored in a storage medium such as the HD 204, for example.

The first prediction unit 2001 performs a first prediction process of predicting the motivation indicators of the user 101 based on the work information of the user 101. For example, the first prediction unit 2001 inputs the work information of the user 101 in prediction models 414 previously created with the work information of a plurality of subjects as an explanatory variable and the motivation indicators of the subjects as objective variables, to thereby predict the motivation indicators of the user 101. The first prediction unit 2001 further stores the predicted motivation indicators of the user 101 in the storage unit 408, for example.

The second prediction unit 2002 performs a second prediction process of predicting the condition indicators of the user 101 based on the work information of the user 101. For example, the second prediction unit 2002 inputs the work information of the user 101 in prediction models 414 previously created with the work information of a plurality of subjects as an explanatory variable and the condition indicators of the subjects as objective variable, to thereby predict the condition indicators of the user 101. The second prediction unit 2002 further stores the predicted condition indicators of the user 101 in the storage unit 408, for example.

Thereby, the calculation unit 404 of the third embodiment calculates the indicators indicating the performance of the user 101 with the motivation indicators and the condition indicators of the user 101 stored in the storage unit 408, for example.

The processes of the factor analysis unit 405 and the output unit 407 of the third embodiment may be similar to those of the first embodiment.

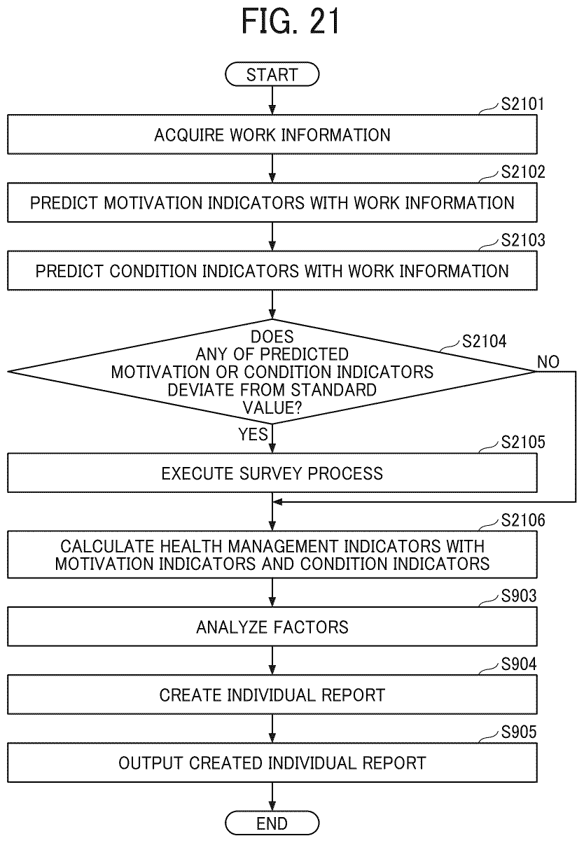

FIG. 21 is a flowchart illustrating an example of an individual report output process of the third embodiment. Specifically, FIG. 21 illustrates an example of an individual report output process performed by the information processing apparatus 100 of the third embodiment described above with FIG. 20. The processes of steps S903 and S905 in the individual report output process of FIG. 21 are similar to those of the individual report output process of the first embodiment described above with FIG. 9A, and thus description thereof will be omitted here.

At step S2101, the third acquisition unit 1801 acquires the work information of the user 101. For instance, if the latest work information of the user 101 is already stored in the storage unit 408, for example, the third acquisition unit 1801 acquires the work information of the user 101 from the storage unit 408. If the latest work information of the user 101 is not stored in the storage unit 408, for example, the third acquisition unit 1801 acquires the work information of the user 101 from the business operation system 1800.

At step S2102, the first prediction unit 2001 predicts the motivation indicators of the user 101 with the acquired work information of the user 101.

At step S2103, the second prediction unit 2002 predicts the condition indicators of the user 101 with the acquired work information of the user 101.

At step S2104, the calculation unit 404 determines whether any of the predicted motivation indicators or condition indicators of the user 101 deviates from the corresponding standard value. For example, if the difference between the value of one of the predicted motivation indicators and the corresponding standard value is equal to or greater than a first threshold value, or if the difference between the value of one of the predicted condition indicators and the corresponding standard value is equal to or greater than a second threshold value, the calculation unit 404 determines that there is an indicator deviating from the standard value.

If it is determined that there is an indicator deviating from the standard value (YES at step S2104), the calculation unit 404 proceeds to the process of step S2105. If it is determined that there is no indicator deviating from the standard value (NO at step S2104), the calculation unit 404 proceeds to the process of step S2106.

At step S2105, the information processing system I executes the survey process including steps S502 to S506 of FIG. 5, for example. The processes of steps S2104 and S2105 are optional and not required.

At step S2106, the calculation unit 404 calculates the health management indicators with the motivation indicators and the condition indicators of the user 101.

With the individual report output process of FIG. 21, the information processing apparatus 100 predicts the health management indicators of the user 101 based on the work information of the user 101, for example, without executing the survey process.

Further, if any of the motivation indicators or the condition indicators of the user 101 predicted from the work information deviates from the standard value, the information processing apparatus 100 may execute the survey process to more accurately predict the indicators indicating the performance of the user 101.

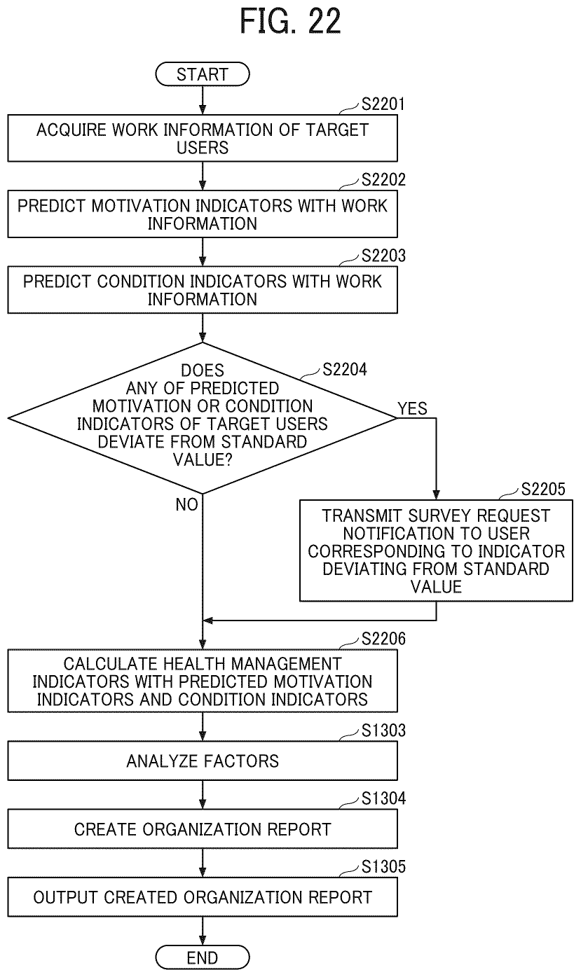

FIG. 22 illustrates an example of an organization report output process of the third embodiment. Specifically, FIG. 22 illustrates an example of an organization report output process performed by the information processing apparatus 100 of the third embodiment described above with FIG. 20. The processes of steps S1303 to S1305 in the organization report output process of FIG. 22 are similar to those of the organization report output process of the first embodiment described above with FIG. 13A, and thus description thereof will be omitted here.

At step S2201, the third acquisition unit 1801 acquires the work information of the target users 101. For example, the third acquisition unit 1801 acquires the work information of the users 101a, 101b, 101c, and so forth, who are the members of the organization administered by the administrator 102, from the business operation system 1800 or the storage unit 408, for example.

At step S2202, the first prediction unit 2001 predicts the motivation indicators of the target users 101 with the acquired work information of the target users 101.

At step S2203, the second prediction unit 2002 predicts the condition indicators of the target users 101 with the acquired work information of the target users 101.

At step S2204, the calculation unit 404 determines whether any of the predicted motivation indicators or condition indicators of the target users 101 deviates from the corresponding standard value. If any of the predicted motivation indicators or condition indicators of the target users 101 deviates from the standard value (YES at step S2204), the calculation unit 404 proceeds to the process of step S2205. If none of the predicted motivation indicators or condition indicators of the target users 101 deviates from the standard value (NO at step S2104), the calculation unit 404 proceeds to the process of step S2206.

At step S2205, the survey execution unit 410 transmits the survey request notification to the user 101 corresponding to the indicator deviating from the standard value, and proceeds to the process of step S2206.

At step S2206, the calculation unit 404 calculates the health management indicators with the predicted motivation indicators and condition indicators of the target users 101.

With the organization report output process of FIG. 22, the information processing apparatus 100 predicts the health management indicators of the target users 101 based on the work information of the target users 101, for example, without executing the survey process.

Further, if any of the motivation indicators or condition indicators of the target users 101 predicted from the work information deviates from the standard value, the information processing apparatus 100 prompts the user 101 corresponding to the motivation or condition indicator to execute the survey process.

At least one of the embodiments of the present invention provides the information processing apparatus 100 that visualizes the indicators in consideration of the motivation of the user 101.

As described above, each of the embodiments of the present invention provides the information processing apparatus 100 that visualizes the indicators related to various performance of the personnel.

Further, with the information processing apparatus 100 of one of the embodiments, indicators such as the health management indicators managed by the company are obtained accurately and frequently.

The apparatuses described in any of the embodiments form one of a plurality of computing environments for implementing the embodiment disclosed in the present specification. In an embodiment of the present invention, the information processing apparatus 100 is a server cluster including a plurality of computing devices configured to communicate with each other via a desired type of communication link such as a network or a shared memory to execute the processes disclosed in the present specification. Similarly, the terminal apparatus 10 may include a plurality of computing devices configured to communicate with each other.

Part or all of the functions of the survey execution unit 410 may be executed by the terminal apparatus 10. Further, the components of the information processing apparatus 100 may be integrated in a single apparatus, or may be distributed to a plurality of apparatuses. Furthermore, the components of the information processing apparatus 100 may be included in the terminal apparatus 10. In this case, the terminal apparatus 10 is an information processing apparatus according to an embodiment of the present invention.

The present specification discloses an information processing apparatus, an information processing system, an information processing method, and a non-transitory recording medium according to the following aspects.

According to a first aspect, an information processing apparatus includes a first acquisition unit, a second acquisition unit, a calculation unit, and an output unit. The first acquisition unit acquires information indicating motivation of a user from an answer to an inquiry to the user. The answer has been input to a terminal apparatus. The second acquisition unit acquires information indicating a condition related to health of the user. The information indicating the condition related to the health of the user is acquired from the inquiry with the terminal apparatus. The calculation unit calculates a plurality of indicators associated with an organization based on the information indicating the motivation of the user and the information indicating the condition related to the health of the user. The output unit outputs the plurality of indicators.

According to a second aspect, in the information processing apparatus of the first aspect, the information indicating the condition related to the health of the user includes information indicating a state of an autonomic nervous system of the user, measured with an image of the user captured with an image capturing device built in or connected to the terminal apparatus, which is used by the user.

According to a third aspect, in the information processing apparatus of the first or second aspect, the user is a member of the organization, and the plurality of indicators include a plurality of health management indicators managed by the organization.

According to a fourth aspect, in the information processing apparatus of the third aspect, the calculation unit calculates each of the plurality of health management indicators managed by the organization with a prediction model. The prediction model includes the information indicating the motivation of the user and the information indicating the condition related to the health of the user each as an explanatory variable and including the each of the plurality of health management indicators as an objective variable.

According to a fifth aspect, the information processing apparatus of the third aspect includes a third acquisition unit that acquires work information of the user. The calculation unit calculates each of the plurality of health management indicators managed by the organization with a prediction model. The prediction model includes the information indicating the motivation of the user, the information indicating the condition related to the health of the user, and the work information of the user each as an explanatory variable and the each of the plurality of health management indicators as an objective variable.

According to a sixth aspect, the information processing apparatus of one of the third to fifth aspects includes a setting receiving unit that receives respective degrees of importance of the plurality of health management indicators set by an administrator of the organization. The output unit preferentially outputs a health management indicator of a highest degree of importance out of the plurality of health management indicators.

According to a seventh aspect, the information processing apparatus of the first or second aspect includes a third acquisition unit that acquires work information of the user. The calculation unit calculates the plurality of indicators with the information indicating the motivation of the user, the information indicating the condition related to the health of the user, and the work information of the user.

According to an eighth aspect, the information processing apparatus of the first or second aspect includes a third acquisition unit, a first prediction unit, and a second prediction unit. The third acquisition unit acquires work information of the user. The first prediction unit predicts the information indicating the motivation of the user based on the work information of the user. The second prediction unit predicts the information indicating the condition related to the health of the user based on the work information of the user.

According to a ninth aspect, when a value of the information indicating the motivation of the user and predicted by the first prediction unit or the information indicating the condition related to the health of the user and predicted by the second prediction unit deviates from a standard value of the information, the information processing apparatus of the eighth aspect starts a survey to acquire the information indicating the motivation of the user and the information indicating the condition related to the health of the user.

According to a tenth aspect, in the information processing apparatus of one of the first to ninth aspects, the output unit outputs at least one of a first comparison result or a second comparison result. The first comparison result represents a result of comparison between an actual measurement value and a standard value of the information indicating the motivation of the user. The second comparison result represents a result of comparison between an actual measurement value and a standard value of the information indicating the condition related to the health of the user.

According to an eleventh aspect, in the information processing apparatus of the tenth aspect, the output unit outputs information related to an improvement plan for the motivation of the user corresponding to the first comparison result or for the condition related to the health of the user corresponding to the second comparison result.

According to a twelfth aspect, the information processing apparatus of one of the first to eleventh aspects includes a factor analysis unit that analyzes a plurality of influential factors on each of the plurality of indicators. The output unit outputs, out of the plurality of influential factors, a factor with a highest degree of influence on the each of the plurality of indicators.

According to a thirteenth aspect, in the information processing apparatus of the twelfth aspect, the output unit preferentially outputs the factor with the highest degree of influence on the each of the plurality of indicators.

According to a fourteenth aspect, in the information processing apparatus of the twelfth or thirteenth aspect, the output unit outputs information related to an improvement plan for the factor with the highest degree of influence on the each of the plurality of indicators.

According to a fifteenth aspect, in the information processing apparatus of the first or second aspect, the output unit outputs a display screen to display an indicator of the plurality of indicators. In response to selection of the indicator on the display screen, the output unit displays, on the display screen, the information indicating the motivation of the user or the information indicating the condition related to the health of the user causally related to the selected indicator.

According to a sixteenth aspect, in the information processing apparatus of the fifteenth aspect, the output unit displays a plurality of indicators of the plurality of indicators on the display screen. In response to selection of the plurality of indicators from the plurality of indicators on the display screen, the output unit preferentially displays, on the display screen, the information indicating the motivation of the user or the information indicating the condition related to the health of the user causally related to the selected plurality of indicators.

According to a seventeenth aspect, in the information processing apparatus of the fifteenth aspect, the output unit preferentially displays, on the display screen, one of the information indicating the motivation of the user and the information indicating the condition related to the health of the user causally related to the selected indicator and having a value that deviates from a standard value more than the other one of the information indicating the motivation of the user and the information indicating the condition related to the health of the user

According to an eighteenth aspect, an information processing system includes a terminal apparatus used by a user and an information processing apparatus communicable with the terminal apparatus. The information processing apparatus includes a first acquisition unit, a second acquisition unit, a calculation unit, and an output unit. The first acquisition unit acquires information indicating motivation of the user from an answer to an inquiry to the user. The answer has been input to the terminal apparatus. The second acquisition unit acquires information indicating a condition related to health of the user. The information indicating the condition related to the health of the user is acquired from the inquiry with the terminal apparatus. The calculation unit calculates a plurality of indicators associated with an organization based on the information indicating the motivation of the user and the information indicating the condition related to the health of the user. The output unit outputs the plurality of indicators.

According to a nineteenth aspect, an information processing system includes a non-transitory recording medium and an information processing apparatus. The non-transitory recording medium stores a plurality of instructions executed by one or more processors of a terminal apparatus used by a user. The information processing apparatus is communicable with the terminal apparatus. The information processing apparatus includes a first acquisition unit, a second acquisition unit, a calculation unit, and an output unit. The first acquisition unit acquires information indicating motivation of the user from an answer to an inquiry to the user. The answer has been input to the terminal apparatus. The second acquisition unit acquires information indicating a condition related to health of the user. The information indicating the condition related to the health of the user is acquired from the inquiry with the terminal apparatus. The calculation unit calculates a plurality of indicators associated with an organization based on the information indicating the motivation of the user and the information indicating the condition related to the health of the user. The output unit outputs the plurality of indicators. When executed by the one or more processors of the terminal apparatus, the plurality of instructions cause the one or more processors to perform a process of transmitting the information indicating the motivation of the user to the information processing apparatus.

According to a twentieth aspect, an information processing method performed by a computer includes a process of acquiring information indicating motivation of a user from an answer to an inquiry to the user, having been input to a terminal apparatus, a process of acquiring information indicating a condition related to health of the user from the inquiry with the terminal apparatus, a process of calculating a plurality of indicators associated with an organization based on the information indicating the motivation of the user and the information indicating the condition related to the health of the user, and a process of outputting the plurality of indicators.

According to a twenty-first aspect, a non-transitory recording medium stores a plurality of instructions which, when executed by one or more processors of a computer, cause the one or more processors to perform an information processing method. The information processing method includes a process of acquiring information indicating motivation of a user from an answer to an inquiry to the user, having been input to a terminal apparatus, a process of acquiring information indicating a condition related to health of the user from the inquiry with the terminal apparatus, a process of calculating a plurality of indicators associated with an organization based on the information indicating the motivation of the user and the information indicating the condition related to the health of the user, and a process of outputting the plurality of indicators.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application specific integrated circuits (ASICs), digital signal processors (DSPs), field programmable gate arrays (FPGAs), conventional circuitry and/or combinations thereof which are configured or programmed to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

1. An information processing apparatus comprising circuitry configured to
acquire first information from an answer to an inquiry to a user, having been input to a terminal apparatus used by the user, the first information indicating motivation of the user,
acquire second information from the inquiry with the terminal apparatus, the second information indicating a condition related to health of the user,
calculate a plurality of indicators associated with an organization based on the first information and the second information, and
output the plurality of indicators.

2. The information processing apparatus of claim 1, wherein the second information includes information indicating a state of an autonomic nervous system of the user, measured with an image of the user captured with an image capturing device built in or connected to the terminal apparatus.

3. The information processing apparatus of claim 1, wherein the user is a member of the organization, and
wherein the plurality of indicators include a plurality of health management indicators managed by the organization.

4. The information processing apparatus of claim 3, wherein the circuitry calculates each of the plurality of health management indicators with a prediction model, the prediction model including the first information and the second information each as an explanatory variable and including the each of the plurality of health management indicators as an objective variable.

5. The information processing apparatus of claim 3, wherein the circuitry
acquires work information of the user, and
calculates each of the plurality of health management indicators with a prediction model, the prediction model including the first information, the second information, and the work information of the user each as an explanatory variable and including the each of the plurality of health management indicators as an objective variable.

6. The information processing apparatus of claim 3, wherein the circuitry
receives respective degrees of importance of the plurality of health management indicators set by an administrator of the organization, and
preferentially outputs a health management indicator of a highest degree of importance out of the plurality of health management indicators.

7. The information processing apparatus of claim 1, wherein the circuitry
acquires work information of the user, and
calculates the plurality of indicators with the first information, the second information, and the work information of the user.

8. The information processing apparatus of claim 1, wherein the circuitry
acquires work information of the user,
predicts the first information based on the work information of the user, and
predicts the second information based on the work information of the user.

9. The information processing apparatus of claim 8, wherein when a value of the predicted first information deviates from a standard value of the first information, the circuitry starts a survey to acquire the first information and the second information.

10. The information processing apparatus of claim 8, wherein when a value of the predicted second information deviates from a standard value of the second information, the circuitry starts a survey to acquire the first information and the second information.

11. The information processing apparatus of claim 1, wherein the circuitry outputs at least one of a first comparison result or a second comparison result,
the first comparison result representing a result of comparison between an actual measurement value and a standard value of the first information, and
the second comparison result representing a result of comparison between an actual measurement value and a standard value of the second information.

12. The information processing apparatus of claim 11, wherein the circuitry outputs information related to an improvement plan for the motivation of the user corresponding to the first comparison result or for the condition related to the health of the user corresponding to the second comparison result.

13. The information processing apparatus of claim 1, wherein the circuitry
analyzes a plurality of influential factors on each of the plurality of indicators, and
outputs, out of the plurality of influential factors, a factor with a highest degree of influence on the each of the plurality of indicators.

14. The information processing apparatus of claim 13, wherein the circuitry preferentially outputs the factor with the highest degree of influence on the corresponding indicator of the plurality of indicators.

15. The information processing apparatus of claim 14, wherein the circuitry outputs information related to an improvement plan for the factor with the highest degree of influence on the corresponding indicator of the plurality of indicators.

16. The information processing apparatus of claim 1, wherein the circuitry
outputs a display screen to display an indicator of the plurality of indicators, and
in response to selection of the indicator on the display screen, displays, on the display screen, the first information or the second information causally related to the selected indicator.

17. The information processing apparatus of claim 16, wherein the circuitry
displays a plurality of indicators of the plurality of indicators on the display screen, and
in response to selection of the plurality of indicators from the plurality of indicators on the display screen, preferentially displays, on the display screen, the first information or the second information causally related to the selected plurality of indicators.

18. The information processing apparatus of claim 16, wherein the circuitry
preferentially displays, on the display screen, one of the first information and the second information causally related to the selected indicator and having a value that deviates from a standard value more than the other one of the first information and the second information.

19. An information processing system comprising:
a terminal apparatus including first circuitry, the terminal apparatus being used by a user; and
an information processing apparatus including second circuitry, the information processing apparatus being communicable with the terminal apparatus,
the first circuitry being configured to
transmit first information to the information processing apparatus, the first information indicating motivation of the user and being acquired from an answer to an inquiry to the user, having been input to the terminal apparatus, and
the second circuitry being configured to
acquire the first information from the terminal apparatus,
acquire second information from the inquiry with the terminal apparatus, the second information indicating a condition related to health of the user,
calculate a plurality of indicators associated with an organization based on the first information and the second information, and
output the plurality of indicators.

20. An information processing method performed by a computer, the information processing method comprising:
acquiring first information from an answer to an inquiry to a user, having been input to a terminal apparatus, the first information indicating motivation of the user;
acquiring second information from the inquiry with the terminal apparatus, the second information indicating a condition related to health of the user;
calculating a plurality of indicators associated with an organization based on the first information and the second information; and
outputting the plurality of indicators.
